(19) United States
(12) Patent Application Publication
(10) Pub. No.: US 2014/0249413 A1
(43) Pub. Date: Sep. 4, 2014

(54) THERANOSIS OF MACROPHAGE-ASSOCIATED DISEASES WITH ULTRASMALL SUPERPARAMAGNETIC IRON OXIDE NANOPARTICLES (USPIO)

(71) Applicants: Gerald L. Wolf, Newton, MA (US); Karl F. Schmidt, Watertown, MA (US)

(72) Inventors: Gerald L. Wolf, Newton, MA (US); Karl F. Schmidt, Watertown, MA (US)

(21) Appl. No.: 14/202,044

(22) Filed: Mar. 10, 2014

Related U.S. Application Data
(63) Continuation of application No. 13/920,694, filed on Jun. 18, 2013.
(60) Provisional application No. 61/690,005, filed on Jun. 18, 2012.
(60) Provisional application No. 61/690,006, filed on Jun. 18, 2012.
(60) Provisional application No. 61/742,382, filed on Aug. 9, 2012.
(60) Provisional application No. 61/743,428, filed on Sep. 4, 2012.
(60) Provisional application No. 61/797,757, filed on Dec. 14, 2012.
(60) Provisional application No. 61/779,123, filed on Mar. 13, 2013.

(51) Int. Cl. A61K 49/18; A61B 17/32

(52) U.S. Cl. CPC A61K 49/1818; A61B 17/320068
USPC 600/439; 424/9.32; 600/476

(57) ABSTRACT

Macrophages sequester and aggregate ultrasmall superparamagnetic iron oxide nanoparticles (USPIO) in their lysosomes. The amount of USPIO loading of macrophages depends upon the route and dose of administration, and the pharmacokinetics of accumulation and removal. Both fixed macrophages and activated macrophages associated with inflammatory diseases and cancer phagocytize USPIOs, and the loaded macrophages can serve to identify the extent of a macrophage-dependent disease as well as to direct treatment options. Furthermore, the absorption of energy from incident electromagnetic waves by the aggregated nanoparticles can be used for conformal thermotherapy. The USPIOs can further be used to carry drugs to the same activated macrophages. The co-administered drugs can be bound to the USPIO by condition-dependent releasing links that are responsive to local pH or heating.

### REFERENCE TO RELATED APPLICATIONS

This application is a continuation of U.S. Utility application Ser. No. 13/920,694, filed on Jun. 18, 2013, which claims the benefit of the filing date under 35 U.S.C. 119(e) of U.S. Provisional Application No. 61/690,005, filed on Jun. 18, 2012; U.S. Provisional Application No. 61/690,006, filed on Jun. 18, 2012; U.S. Provisional Application No. 61/742,382, filed on Aug. 9, 2012; U.S. Provisional Application No. 61/743,428, filed on Sep. 4, 2012; U.S. Provisional Application No. 61/797,757, filed on Dec. 14, 2012; and U.S. Provisional Application No. 61/779,123, filed on Mar. 13, 2013. The entire content of each of these applications, including drawings, are incorporated herein by reference.

### BACKGROUND OF THE INVENTION

Medical imaging is the technique and process for creating images of the live human body or parts thereof for clinical purposes, most notably for diagnostic uses or for medical science research. Over the years, numerous medical imaging devices and technologies have been developed based on distinct scientific principles that incorporate radiology, nuclear medicine, investigative radiological sciences, endoscopy, medical thermography, medical photography, and microscopy. To date, the most widely used medical imaging technologies utilize the effect of tissues upon the transmission, scattering, or absorption of energy. The energy can take the form of ionizing radiation, as used for numerous x-ray techniques, such as plain films; or computed tomography (CT scan, or Computed Axial Tomography or CAT scan); or nuclear medicine, techniques where the radiation is emitted by radioactive isotopes as used in scintigraphy, Single Photon Emission Tomography (SPECT), or Positron Emission Tomography (PET). However, these forms of radiation do carry a potential burden of causing adverse effects when there energetic photons damage the body's genes. In addition, macrophages do not have special properties that make them visible with these forms of radiation, and there are currently no good biomarkers that can be used to selectively image macrophages with this form of radiation. Some less harmful energy forms may be used to image the macrophages.

Magnetic Resonance Imaging (MRI), also known as nuclear magnetic resonance imaging (NMRI), or magnetic resonance tomography (MRT) is a medical imaging technique used to visualize internal structures of the body. Body tissue contains large amounts of water (H₂O), and hence protons (¹H nuclei) since each water molecule has two hydrogen nuclei (protons). The protons can be aligned in a strong magnetic field generated by a powerful magnet of an MRI scanner. When a person is inside this magnetic field, the average magnetic moment of many protons becomes aligned with the direction of the field. A radio frequency current is briefly turned on, producing a varying electromagnetic field. This electromagnetic field has just the right frequency, known as the resonance frequency, to be absorbed and flip the spin of the protons in the magnetic field. After the electromagnetic field is turned off, the spins of the protons return to thermodynamic equilibrium and the bulk magnetization becomes re-aligned with the static magnetic field. During this relaxation, a radio frequency signal (electromagnetic radiation in the RF range) is generated, which can be recorded and measured with receiver coils of the MRI scanner. The recorded information is used to construct an image of the scanned area of the body. The energy absorbed by the body during MRI is small, and it is thought that MRI can be used safely and repeatedly for diagnosis.

MRI provides good contrast between the different soft tissues of the body and can be used to image every part of the body, particularly for tissues with many hydrogen nuclei and little density contrast, such as the brain, muscle, connective tissue and most tumors, because the composition of these tissues influences the relaxation of the protons which are imaged. However, macrophages look quite similar to surrounding tissues when imaged with MRI.

To enhance the appearance of blood vessels, tumors or inflammation in MRI, certain MRI contrast agents may be injected intravenously. MRI contrast agents are a group of contrast media or biomarkers used to improve the visibility of internal body structures in MRI. They alter the relaxation times of atoms within body tissues wherever they are present after administration.

Gadolinium (III) containing contrast agents are the most commonly used MRI biomarkers for enhancement of vessels in MR angiography or for brain tumor enhancement associated with the degradation of the blood-brain barrier. Without attachment to large targeting molecules, these common MR contrast agents do not accumulate in cells, including macrophages.

The other type of MRI biomarker is iron oxide based contrasting agents, which include superparamagnetic iron oxide (SPIO) and ultrasmall superparamagnetic iron oxide (USPIO). These contrast agents consist of suspended colloids of magnetizable iron oxide nanoparticles, and, when injected, increase the relaxation of nearby protons in ways that are clearly imageable by the scanner and its programs.

MRI contrast agents may be administered by injection into the blood stream or orally. Oral administration is well suited to G.I. tract scans, while intravascular administration may be more useful for most other scans.

Medical ultrasonography (or simply “ultrasound”) uses high frequency broadband sound waves in the megahertz range that are reflected by tissue to varying degrees to produce up to 3D images. Ultrasound is an oscillating sound pressure wave with a frequency greater than the upper limit of the human hearing range. Thus ultrasound is not separated from audible sound based on differences in physical properties, but only on the fact that humans cannot hear it. Although this limit varies from person to person, it is about 20 kilohertz (20,000 Hz) in healthy, young adults.

Ultrasound devices operate with frequencies from 20 kHz (2×10⁴ Hz) up to several gigahertz (1×10⁹ Hz). Ultrasonic imaging typically uses frequencies of 2 megahertz (2×10⁶ Hz) and higher—the shorter wavelength allows resolution of small internal details in structures and tissues. A 3 GHz sound wave can produce an image resolution comparable to that of an optical image. The power density is generally less than 1 watt per square centimeter, in order to avoid heating and cavitation effects in the object under examination. Thus, ultrasound used for imaging is considered quite safe.

Ultrasound imaging is commonly associated with imaging the fetus in pregnant women, but is also broadly used in, for example, imaging the abdominal organs, heart, breast, muscles, tendons, arteries and veins. While it may provide less anatomical detail than techniques such as CT or MRI, it has several advantages which make it ideal in numerous situations, in particular that it studies the function of moving structures in real-time, and emits no ionizing radiation, and contains speckle that can be used in elastography (a non-invasive method in which stiffness or strain images of soft tissue are used to detect or classify tumors, based on the fact that a tumor or a suspicious cancerous growth is normally 5-28 times stiffer than the background of normal soft tissue. Thus when a mechanical compression or vibration is applied, the tumor deforms less than the surrounding tissue).

Ultrasound is relatively inexpensive and quick to perform. Ultrasound scanners are portable and can be taken to the patient's location. Although there are ultrasound contrast agents composed of gas bubbles that are readily visualized, these agents are relatively large with sizes hundreds of times larger than USPIOs and their persistence time is measure in minutes. They are not currently useful for imaging TAMs or IAMs.

Optical Coherence Tomography, or “OCT,” is a technique for obtaining sub-surface images of translucent or opaque materials at a resolution equivalent to a low-power microscope. It images light wave reflections from within tissue to provide cross-sectional images.

During OCT, an optical beam is directed at the tissue, and a small portion of this light that reflects from sub-surface features is collected. Most light, however, is not reflected but scatters off at large angles. In conventional imaging, this diffusely scattered light contributes background noise that obscures an image. However, in OCT, a technique called interferometry is used to record the optical path length of received photons allowing rejection of most photons that scatter multiple times before detection. Thus OCT can build up clear 3D images of thick samples by rejecting background signal while collecting light directly reflected from surfaces of interest. The technique, however, is limited to imaging 1 to 2 mm below the surface in biological tissue, because at greater depths, the proportion of light that escapes without scattering is too small to be detected. In order to distinguish tissues by means other than their location, special fluorescent chemicals or photodynamic agents must be given.

Cellular phagocytosis is an ancient capability for some cells, perhaps appearing in evolution as early as the amoeba. Common to more advanced species, macrophages have evolved numerous receptors on their cell membranes to discriminate what materials constitute a threat to the organism and should be ingested. In this way, macrophages serve as an internal defense against pathogens.

Macrophages can be divided into normal or inflammatory macrophages. The former are also consider fixed or sessile, and are normally present within the body organs where they have been named Kuppfer cells (liver), histiocytes (muscle), dendritic cells (skin), or just identified by location (e.g., spleen, lymph node, alveoli, adrenal, or bone marrow macrophages). Thus such macrophages are also referred to as “tissue macrophages” as used herein.

On the other hand, inflammation is characterized by local accumulation of cells from the blood, including monocytes—the precursors of macrophages, and inflammatory macrophages (also known as activated macrophages, or Inflammation Associated Macrophages (IAMs) invade diseased tissues in response to a “danger signal.” See Matzinger (Science 296:302-305, 2002). Under normal circumstances, inflammatory reactions are part of the host defense, playing a critical role in the eradication of infectious agents and removing debris. However, inflammatory processes are intrinsically destructive to normal tissues, and they can in certain circumstances do far more harm than good. Thus, activated macrophages play a key pathophysiologic role in many common inflammatory diseases, disorders or conditions, including primary and metastatic tumors. See Sica (European J. Cancer 42:717-727, 2006).

### BRIEF SUMMARY OF THE INVENTION

Accurate diagnosis and selective therapy for inflammatory diseases has not previously been achieved with a theranostic approach. Yet, these diseases share certain pathophysiologic processes which can be imaged and subsequently treated based upon the activities of macrophages that are key cellular components for such diseases. Macrophage-dependent or associated diseases include primary and metastatic cancer, vulnerable arterial plaque, chronic obstructive pulmonary disease, periodontal disease, rheumatoid arthritis, and many more. The macrophages that play a key role in aggressive cancers are called tumor-associated macrophages (TAMs), whereas the macrophages associated with other inflammatory diseases are properly called Inflammation-associated Macrophages (IAMs).

Under the proper conditions, these macrophages can be imaged and then used for selective treatment because they accumulate and aggregate ultrasmall superparamagnetic iron oxide nanoparticles (USPIOs). It is Applicants' surprising realization that USPIOs can serve as macrophage-enhancing contrast agents for medical imaging devices other than MRI, e.g., ultrasound and OCT, that provides new diagnostic opportunities for inflammatory diseases. Applicants further realized that the same USPIOs already aggregated within the TAMs or IAMs can be used for selective therapy. That is, USPIOs can be used for both diagnosis and treatment, i.e., “theranosis,” in a wide range of macrophage-dependent diseases.

Thus the present invention relates to the surprising use of ultrasmall superparamagnetic iron oxide (USPIO) nanoparticles for detecting tissue or activated macrophages (particularly useful in activated macrophages) after phagocytosis and aggregation of the USPIO nanoparticles within such macrophages, e.g., under conditions or wavelengths suitable for detecting Rayleigh or Mie scattering.

Both Rayleigh and Mie scattering emphasize the dependence on the ratio between particle size and the imaging wavelength. USPIO nanoparticles of about 25 nm would be relatively inefficient contrast agents at wavelengths used for ultrasonic imaging devices or for optical coherent tomography devices. In either case, within the sensitive scattering region for both devices, scattering increases dramatically with increased particle size, such as the enlarged size (typically >100 nm, or more than 500 nm, or up to about 2 μm) of the aggregates formed inside the lysosomal/endosomal compartment of the macrophage that has phagocytized the USPIO nanoparticles. See Levy (infra). While not wishing to be bound by any particular theory, Applicants believe that other mechanisms, such as acoustic or optical mismatches, may further contribute to detectable changes in electromagnetic wave interactions within the loaded macrophages, since cells do not normally contain large amounts of inorganic particles.

The present invention also relates to the treatment of disease, disorder, or conditions associated with USPIO-loaded macrophages based on, for example, the ability of the phagocytosed and aggregated USPIO nanoparticles within the macrophages to serve as a heat sink or drug depot that can release drug under controlled conditions (such as heating, digestion, and/or pH change).

Fixed tissue macrophages and disease-associated activated macrophages readily ingest USPIOs from nearby body fluids. Useful USPIO formulations consist of a magnetizable iron oxide core and a biocompatible coating. For presentation to macrophages in macrophage-dependent or -associated diseases, the USPIO should have a suitable size to facilitate extravasation from the enhanced capillary permeability and interstitial diffusion to the disease location, where they are readily ingested and stored in lysosomes. This active process creates nanoparticle aggregates of much larger size, resulting in increased scattering and absorption of incident electromagnetic waves, including those of light (e.g., laser), radiofrequency (RF), microwave (MW), and ultrasound (US). As a result, the location and density of macrophages with the aggregated USPIO nanoparticles can be imaged, and the presence and severity of disease in that location effectively staged. Thus, the scattering and absorption from aggregated particles create different imaging mechanisms than that associated with the use of USPIO nanoparticles as MRI contrast agents.

Furthermore, these focal USPIO aggregates can be locally heated with hyperthermic devices, and the heat absorption will conform to the distribution of the activated macrophages, which closely mirrors the geometry of the tissue to be treated. During imaging or heating, the USPIO-loaded macrophages do not have to be (e.g., is not) subjected to any magnetic field, e.g., any static or time-varying magnetic field. In addition, the USPIO coat can be used to deliver useful payloads of bound drug to the diseased site, and suitable spacers and linkers can also provide for facilitated release due to, for example, the lysosomal pH or applied local heat.

Thus, the invention provides USPIO-based technology that can be used for both diagnosis and therapy—“theranosis” of macrophage-dependent or -associated diseases. The local USPIO content in macrophage-dependent or -associated diseases is responsive to route of administration, dose, and pharmacokinetics. The imaging and treatment applications can be monitored and repeated as indicated. The resulting theranosis is responsive to the extant pathophysiology in the subject, and may constitute personalized medicine for that subject.

Thus one aspect of the invention provides a method for detecting activated macrophages in a subject, wherein the subject has a disease or condition associated with the activated macrophages, the method comprising: (1) administering to the subject a formulation of ultrasmall superparamagnetic iron oxide (USPIO) nanoparticles having an average size of about 10-50 nm (e.g., about 20-50 nm, about 20-40 nm, about 20-30 nm, about 25 nm); (2) waiting for a pre-determined time (e.g., 30 min to about 14 days, depending on administration route, dose, and repeated administration) to allow the USPIO nanoparticles to accumulate as aggregates inside the activated macrophages, wherein the aggregates have an average size of greater than 100 nm (or 500 nm, or 1 μm, or 2 μm); and, (3) imaging the activated macrophages with a medical imaging device (such as an ultrasound or optical imaging device) that produces an image based at least in part on Rayleigh and/or Mie scattering by the aggregates.

In certain embodiments, the method further comprises assessing the location, density, and/or degree of locally enhanced activated macrophages, thereby staging the macrophage associated disease or condition.

In certain embodiments, the method further comprises evaluating the image, and optionally the clinical circumstances, to determine (1) the need for treatment, (2) treatment options using the USPIO-enhanced macrophages for treating the disease existing at one or more sites comprising said activated macrophages, at the time of evaluation, and/or (3) determining the need for further USPIO dosing regimens to make such treatments feasible.

Another aspect of the invention provides a method for treating a disease or condition associated with activated macrophages, in a subject in need thereof, the method comprising: (1) administering to the subject a formulation of an ultrasmall superparamagnetic iron oxide (USPIO) nanoparticles, wherein the USPIO nanoparticles have an average size of about 10-50 nm (e.g., about 20-50 nm, about 20-40 nm, about 20-30 nm, about 25 nm); (2) waiting for a pre-determined time (e.g., 30 min to about 14 days, depending on administration route, dose, and repeated administration) to allow the USPIO nanoparticles to accumulate as aggregates in the activated macrophages, wherein the aggregates have an average size of greater than 100 nm (or 500 nm, or 1 μm, or 2 μm); (3) locating the activated macrophages with a medical imaging device to identify the size, shape, and/or location of tissues affected by the disease or condition; (4) raising temperature of (e.g., heating) the aggregates by directing an energy that is absorbed by the aggregates, and sustaining the elevated temperature for a time sufficient to kill the cells in tissues affected by the disease or condition and surrounding said activated macrophage.

In certain embodiments, step (3) can be any suitable medical imaging device including MRI.

In certain embodiments, in step (3), the medical imaging device produces an image based at least in part on Rayleigh and/or Mie scattering by the aggregates.

In certain embodiments, the USPIO nanoparticles are coated with a therapeutic agent effective for treating said disease or condition.

In certain embodiments, the therapeutic agent is released upon entering a low pH environment within the activated macrophage.

In certain embodiments, the therapeutic agent is released upon raising the temperature of the aggregates.

In certain embodiments, the energy is high intensity ultrasound energy (e.g., one delivered by a high intensity ultrasound energy device operating with frequencies between 2 and 60 MHz).

In certain embodiments, the energy is radiofrequency (RF) energy (e.g., one delivered by a (medically) approved device utilizing radiofrequency wavelengths with a frequency within the ISM band for such devices).

In certain embodiments, the energy is laser energy (e.g., one delivered by a laser approved for medical treatments and operating within the ultraviolet, visible, and infrared frequencies).

In certain embodiments, the energy is microwave energy (e.g., one delivered by an approved microwave device with a frequency within the ISM band for such devices).

In certain embodiments, the method further comprises assessing macrophage density during therapeutic intervention in order to determine the desirable continuation, change, or cessation of a particular therapy.

In certain embodiments, the USPIO nanoparticles have an average hydrodynamic particle size of about 15-50 nm, or 20-25 nm as determined by dynamic light scattering.

In certain embodiments, the USPIO nanoparticles are sufficiently small to extravasate and diffuse through restricted extracellular matrix surrounding said activated macrophages.

In certain embodiments, the formulation of USPIO nanoparticles is administered to the subject percutaneously, intravenously, by inhalation or ingestion, or otherwise into a body cavity connected to the outside (e.g., rectal, oral, spraying into the nostrils or respiratory tract).

In certain embodiments, the formulation of USPIO nanoparticles is administered to the subject: (1) intravenously at a dose of about 0.5 to 20 mg/kg, optionally repeated (as necessary) with a waiting time between 12 and 144 hours or longer; (2) interstitially (for nodal enhancement) at a dose of about 0.01-2 mg/kg, with stimulation of lymphatic uptake as feasible, and, optionally repeated (as necessary) with a waiting time of between 30 min-14 days; or, (3) intracavitarily at a dose of about 0.05-2 mg/kg (in an appropriate suspension), and waiting 30 min-14 days before imaging.

In certain embodiments, the macrophage-associated disease or condition comprises: primary or metastatic cancer, vulnerable plaque, rheumatoid arthritides, inflammatory bowel disease, chronic obstructive pulmonary disease, bronchial asthma, periodontal disease, or transplant rejection.

In certain embodiments, the macrophage-associated disease or condition is an “inflammatory disease, disorder, or otherwise abnormal condition,” which may include disorders associated with inflammation or have an inflammation component, such as, but are not limited to: acne vulgaris, asthma, autoimmune diseases, celiac disease, prostatitis, glomerulonephritis, hypersensitivities, Crohn's disease, ulcerative colitis, pelvic inflammatory disease, reperfusion injury, sarcoidosis, transplant rejection, vasculitis, interstitial cystitis, atherosclerosis, allergies (type 1, 2, and 3 hypersensitivity, hay fever), inflammatory myopathies, as systemic sclerosis, and include dermatomyositis, polymyositis, inclusion body myositis, Chediak-Higashi syndrome, chronic granulomatous disease, Vitamin A deficiency, periodontitis, Granulomatous inflammation (tuberculosis, leprosy, and syphilis), fibrinous inflammation, purulent inflammation, serous inflammation, ulcerative inflammation, and ischemic heart disease, type I diabetes, and diabetic nephropathy.

In certain embodiments, the inflammatory disease, disorder, or otherwise abnormal condition includes many autoimmune diseases or disorders that are associated with inflammation or have an inflammation component, e.g., corresponding to one or more types of hypersensitivity. Exemplary autoimmune diseases or disorders that correspond to one or more types of hypersensitivity include: atopic allergy, atopic dermatitis, autoimmune hemolytic anemia, autoimmune hepatitis, autoimmune polyendocrine syndrome, autoimmune urticaria, celiac disease, cold agglutinin disease, contact dermatitis, Crohn's disease, diabetes mellitus type 1, discoid lupus erythematosus, Erythroblastosis fetalis, Goodpasture's syndrome, Graves' disease, Guillain-Barré syndrome (GBS), Hashimoto's encephalopathy, Hashimoto's thyroiditis, idiopathic thrombocytopenic purpura, autoimmune thrombocytopenic purpura, IgA nephropathy, lupus erythematosus, Ménière's disease, multiple sclerosis, myasthenia gravis, narcolepsy, neuromyelitis optica, Devic's disease, neuromyotonia, ocular cicatricial pemphigoid, opsoclonus myoclonus syndrome, PANDAS (pediatric autoimmune neuropsychiatric disorders associated with streptococcus), paraneoplastic cerebellar degeneration, pemphigus vulgaris, pernicious anaemia, psoriasis, psoriatic arthritis, rheumatoid arthritis, rheumatic fever, sarcoidosis, scleroderma, subacute bacterial endocarditis (SBE), systemic lupus erythematosis, Lupus erythematosis, temporal arteritis (also known as “giant cell arteritis”), thrombocytopenia, ulcerative colitis, undifferentiated connective tissue disease, urticarial vasculitis, and vasculitis.

In certain embodiments, the pre-determined time (e.g., 30 min to 14 days) is sufficient to allow the USPIO nanoparticles to accumulate as aggregates of at least about 100 nm in size, or 1 μm in size, or 1.5 μm in size, or 2 μm in size, preferably occupying 5-80% of the macrophage cell volume.

In certain embodiments, the method further comprises controlling the size of the aggregates by dose, administration route, waiting time, and frequency of the USPIO formulation administered to said subject.

In certain embodiments, the iron core of the USPIO nanoparticles are coated by a biocompatible polymer (e.g., PEG).

In certain embodiments, the medical imaging device is MRI (e.g., Macrophage-Enhanced MEMRI), ultrasound (e.g., Macrophage-Enhanced ultrasound—MEUS), or optical imaging (e.g., Macrophage-Enhanced Optical—MEOCT).

In certain embodiments, the medical imaging device is not an MRI device, or an OCT device.

In certain embodiments, the macrophage-associated disease or condition comprises primary or metastatic cancer, and cancerous tissue is removed from the subject based on the MEUS or MEOCT images.

In certain embodiments, the macrophage-associated disease or condition comprises primary or metastatic cancer, and the interventional device is positioned based on MEUS or MEOCT images.

In certain embodiments, the macrophage-associated disease or condition comprises primary or metastatic cancer, and biopsy or surgical intervention (e.g., surgical intervention with minimally invasive devices) is performed based on the MEUS or MEOCT images.

Another aspect of the invention provides imagine devices that can be used to carry out the methods of the invention.

In certain embodiments, the device may comprise a portion (e.g., an imaging portion) that can be used to produce a medical image based on the methods of the invention, e.g., imaging the activated macrophages based at least in part on Rayleigh and/or Mie scattering by the aggregates formed from phagocytosed USPIO nanoparticles; and a portion (e.g., a heating portion) that can raise the temperature of such aggregates, based on one or more types of energy source described in any embodiments, such as laser, radio frequency, microwave, or ultrasound.

In certain embodiments, the imaging portion and the heating portion use the same energy form (e.g., both ultrasound or light). In certain embodiments, the imaging portion and the heating portion use different energy forms (e.g., the imaging portion uses ultrasound, while the heating portion uses microwave, RF, or light, etc.).

Other embodiments of the device are as those described in the methods of the invention, and are not reiterated herein.

Unless specified otherwise, doses of the USPIO nanoparticles used herein are expressed as “mg Fe/kg,” or “mg/kg” for short.

Specific embodiments of the invention are further described below in the following numbered paragraphs.
1. A method for detecting activated macrophages in a subject, wherein the subject has a disease or condition associated with said activated macrophages, the method comprising:
   (1) administering to the subject a formulation of ultrasmall superparamagnetic iron oxide (USPIO) nanoparticles having an average size of about 10-50 nm;
   (2) waiting for a pre-determined time to allow the USPIO nanoparticles to accumulate as aggregates inside said activated macrophages, wherein the aggregates have an average size of greater than 100 nm (or 500 nm); and,
   (3) imaging the activated macrophages with a medical imaging device that produces an image based at least in part on Rayleigh and/or Mie scattering by the aggregates.
      optionally, the disease or condition is primary or metastatic cancer; wherein the pre-determined time is about 12-144 hours; wherein the aggregates have an average size of more than 500 nm and up to about 2 μm; wherein the formulation of USPIO nanoparticles is administered to the subject intravenously at a dose of about 0.5 to 20 mg Fe/kg; and wherein the medical imaging device is an ultrasound device.
2. The method of paragraph 1, further comprising assessing the location, density, and/or degree of locally enhanced activated macrophages, thereby staging the macrophage associated disease or condition.
3. The method of paragraph 1 or 2, further comprising evaluating the image to determine (1) the need for treatment, (2) treatment options using the USPIO-enhanced macrophages for treating the disease existing at one or more sites comprising said activated macrophages, at the time of evaluation, and/or (3) determining the need for further USPIO dosing regimens to make such treatments feasible.
4. A method for treating a disease or condition associated with activated macrophages, in a subject in need thereof, the method comprising:
   (1) administering to the subject a formulation of an ultrasmall superparamagnetic iron oxide (USPIO) nanoparticles, wherein the USPIO nanoparticles have an average size of about 10-50 nm;
   (2) waiting for a pre-determined time to allow the USPIO nanoparticles to accumulate as aggregates in said activated macrophages, wherein the aggregates have an average size of greater than 100 nm (or 500 nm);
   (3) locating the activated macrophages with a medical imaging device to identify the size, shape, and/or location of tissues affected by the disease or condition;
   (4) raising temperature of (e.g., heating) the aggregates by directing an energy that is absorbed by the aggregates, and sustaining the elevated temperature for a time sufficient to kill the cells in tissues affected by the disease or condition and surrounding said activated macrophage.
5. The method of paragraph 4, wherein the medical imaging device produces an image based at least in part on Rayleigh and/or Mie scattering by the aggregates
6. The method of paragraph 4, wherein the USPIO nanoparticles are coated with a therapeutic agent effective for treating said disease or condition.
7. The method of paragraph 6, wherein the therapeutic agent is released upon entering a low pH environment within the activated macrophage.
8. The method of paragraph 6, wherein the therapeutic agent is released upon raising the temperature of the aggregates.
9. The method of any one of paragraphs 4-8, wherein the energy is high intensity ultrasound energy (e.g., one delivered by a high intensity ultrasound energy device operating with frequencies between 2 and 60 MHz).
10. The method of any one of paragraphs 4-8, wherein the energy is radiofrequency (RF) energy (e.g., one delivered by a (medically) approved device utilizing radiofrequency wavelengths with a frequency within the ISM band for such devices).
11. The method of any one of paragraphs 4-8, wherein the energy is laser energy (e.g., one delivered by a laser approved for medical treatments and operating within the ultraviolet, visible, and infrared frequencies).
12. The method of any one of paragraphs 4-8, wherein the energy is microwave energy (e.g., one delivered by an approved microwave device with a frequency within the ISM band for such devices).
13. The method of any one of paragraphs 4-12, further comprising assessing macrophage density during therapeutic intervention in order to determine the desirable continuation, change, or cessation of a particular therapy.
14. The method of any one of the preceding paragraphs, wherein the USPIO nanoparticles have an average hydrodynamic particle size of about 15-50 nm as determined by dynamic light scattering.
15. The method of any one of paragraphs 1-14, wherein the USPIO nanoparticles are sufficiently small to extravasate and diffuse through restricted extracellular matrix surrounding said activated macrophages.
16. The method of any one of the preceding paragraphs, wherein the formulation of USPIO nanoparticles is administered to the subject percutaneously, intravenously, by inhalation or ingestion, or otherwise into a body cavity connected to the outside (e.g., rectal, oral, spraying into the nostrils or respiratory tract).
17. The method of any one of the preceding paragraphs, wherein the formulation of USPIO nanoparticles is administered to the subject:
   (1) intravenously at a dose of about 0.5 to 20 mg Fe/kg, optionally repeated (as necessary) with a waiting time between 12 and 144 hours or longer;
   (2) interstitially (for nodal enhancement) at a dose of about 0.01-2 mg Fe/kg, with stimulation of lymphatic uptake as feasible, and, optionally repeated (as necessary) with a waiting time of between 30 min-14 days; or,
   (3) intracavitarily at a dose of about 0.05-2 mg Fe/kg (in an appropriate suspension), and waiting 30 min-14 days before imaging.
18. The method of any one of paragraphs 1-17, wherein the macrophage-associated disease or condition comprises: primary or metastatic cancer, vulnerable plaque, rheumatoid arthritides, inflammatory bowel disease, chronic obstructive pulmonary disease, bronchial asthma, periodontal disease, transplant rejection, or vascular access stenosis.
19. The method of any one of paragraphs 1-18, wherein the pre-determined time is sufficient to allow the USPIO nanoparticles to accumulate as aggregates of at least about 100 nm in size, or 1 μm in size, or 1.5 μm in size, or 2 μm in size, preferably occupying 5-80% of the macrophage cell volume.
20. The method of any one of paragraphs 1-19, further comprising controlling the size of the aggregates by dose, administration route, waiting time, and frequency of the USPIO formulation administered to said subject.
21. The method of any one of paragraphs 1-20, wherein the iron core of the USPIO nanoparticles are coated by a biocompatible polymer (e.g., PEG).
22. The method of any one of the preceding paragraphs, wherein said medical imaging device is ultrasound (e.g., Macrophage-Enhanced ultrasound—MEUS), or optical imaging (e.g., Macrophage-Enhanced Optical—MEOCT).
23. The method of any one of the preceding paragraphs, wherein the medical imaging device is not an MRI device, or an OCT device.
24. The method of paragraph 1, wherein the macrophage-associated disease or condition comprises primary or metastatic cancer, and cancerous tissue is removed from the subject based on the MEUS or MEOCT images.
25. The method of paragraph 1, wherein the macrophage-associated disease or condition comprises primary or metastatic cancer, and the interventional device is positioned based on MEUS or MEOCT images.
26. The method of paragraph 1, wherein the macrophage-associated disease or condition comprises primary or metastatic cancer, and biopsy or surgical intervention (e.g., surgical intervention with minimally invasive devices) is performed based on the MEUS or MEOCT images.

It should be understood that any embodiments described herein, including those described under different aspects of the invention, in different sections of the specification including the Examples, can all be combined with any other embodiments of the invention whenever appropriate.

### BRIEF DESCRIPTION OF THE DRAWINGS

FIG. 1. Schematic representation of the relation between nanoparticle size, electromagnetic wavelength, and scattering.

As the particle size increases, there is an exponential increase in scattering at all the listed wavelengths, from light, radiofrequency (RF), microwave (MW), and ultrasound (US). The relation between nanoparticle size and probing wavelength is shown using Rayleigh and Mie scattering as an example, but additional absorption and reflection processes are also involved. Within the activated macrophages, the ingested nanoparticles (20-30 nm diameter) become aggregates exceeding 500 nm or more in size, leading to clearly increased Rayleigh or Mie scattering.

### DETAILED DESCRIPTION OF THE INVENTION

### 1. Overview

The presentation of ultrasmall superparamagnetic iron oxide (USPIO) nanoparticles to macrophages depends upon the size of the USPIO nanoparticles, their route of administration, and the location of the challenged macrophage.

USPIO particles have originally been developed as contrast agents or biomarkers for MRI. Due to their nature, in general, these particles consist of a solid core of magnetizable iron oxides, and a coating material suitable for the intended use. They can range in size up to 200 nm, but usually are 15-30 nm as measured by in vitro light scattering techniques.

The challenged macrophages may be those normal to a tissue such as liver, spleen, lymph node, lung, intestine, adrenal cortex or bone marrow, or those not normally present in a tissue but representing a part of the pathophysiologic response occurring in that tissue. In both cases, the sequestered nanoparticles increase light scattering of ultrasonic and optical wavelengths from the locally loaded macrophages, thus providing the medical utility of, for example, identifying and/or assessing the local macrophage populations associated with the disease, disorder, or condition. Medical imaging devices that are developed to use radiofrequency or microwave would also be affected by the aggregated USPIO nanoparticles.

The route of USPIO administration also relates to the presentation to the macrophages. When the USPIO nanoparticles are presented in the blood, liver and splenic macrophages can remove them directly as these macrophages line the respective organ blood sinusoids. Lymph nodes, bone marrow, and adrenal macrophages are extravascular, and the USPIO must first extravasate through their fenestrated capillaries. Nanoparticles administered by intracavitary application are taken up by macrophages that have ready access to the adjacent lumen, being separated by only surface coatings. Nanoparticles given interstitially gain access to the terminal lymphatics at that location.

Under normal circumstances, for fixed macrophages, larger nanoparticles such as ferumoxides (e.g., Feridex) are rapidly removed by liver and spleen, and their short circulation time and larger size minimize uptake in the other fixed and inflammatory macrophages. Smaller USPIOs such as ferumoxytol (30 nm) and ferumoxytran-10 (15-30 nm with a median size of 21 nm), when combined with a longer circulation time, do get presented to fixed and inflammatory macrophages where they are readily ingested.

Many diseases, including cancer, elicit chronic inflammation in the body. Despite the great diversity of pathological events that elicit the process, there are common inflammatory responses in the affected tissues. Following local incitement, the affected tissues are invaded by monocytes, perhaps in response to a “danger signal,” and these monocytes then differentiate into activated macrophages. Much of the subsequent inflammatory response is due to the activity of these macrophages. For example, in their activated state, the macrophages release chemokines that increase vascular permeability. See Sica (supra). The presence as well as the changes the activated macrophages induce in the inflamed tissue can be identified and monitored by MRI through the use of biomarkers which assess the resulting increase in local vascular permeability and excess phagocytosis.

One such class of biomarkers, ultrasmall superparamagnetic iron oxide nanoparticles (USPIOs), is able to report both angiogenesis and macrophage infiltration common to such inflammatory diseases. For example, increased vascular permeability, as a result of chemokines produced by the activated macrophages, facilitates escape of the small USPIO nanoparticles from the blood. Once USPIOs are in the extracellular space, interstitial diffusion presents them to the activated macrophages and uptake via phagocytosis is rapid. During phagocytosis, the USPIO particles are transported and sequestered in the lysosomes of the macrophage. There, they remain until metabolized. The loading and retention within the lysosomes create tightly packed aggregates of the ingested USPIOs. The resulting aggregated nanoparticles achieve a size many times larger than the individual nanoparticles—often in the range of hundreds of nanometers. See Levy (Biomaterials 32:3988-3999, 2011). These lysosomes may contain thousands to millions of the original nanoparticles. This aggregation changes the scattering and absorption of electromagnetic waves.

For example, in a macrophage with phagocytosed USPIO nanoparticles, as visualized with TEM, many lysosomes are seen to contain aggregated USPIO nanoparticles (not shown). At higher power, the photomicrograph discerns the tight packing of USPIO nanoparticles, appearing to occupy about 80% of the imaged lysosome.

This basic physiology extends the Enhanced Permeation and Retention (EPR) effect to nanoparticles; however, the retention of USPIO at the inflammatory site is both active and long as contrasted with the EPR benefits for most small chemotherapeutics. The USPIO labeling of the chronic inflammatory site is highly local—usually within several cell diameters of the permeable capillary. This proximity is sometimes referred to as “microscopically intimate.” So long as the activated macrophages are present, extravasated USIOs will accumulate there in close proximity to the tissues that are the source of the danger signal, and this process will continue until the inciting circumstances are eliminated. This is the goal of treatment.

Imaging these common responses with non-invasive MRI and other suitable medical imagining devices can be used to evaluate the seriousness of the disease, its extent, the selection of treatment, the response of the disease to a current treatment regimen, or the recurrence of the disease. The safety of noninvasive imaging, such as MRI, ultrasound or optical imaging, is useful for the many times a patient with macrophage-dependent inflammation could benefit from serial measurement of the extent and activity of his/her disease.

It is important to note that USPIOs generally do not accumulate in non-phagocytic cells within the inflammatory disease unless targeted by added ligands to specific receptors on those cells. In general, targeting diagnostic or therapeutic agents to particular cells has proven difficult and often the targeting ligand, if highly specific, limits the applicability to small cohorts of patients. See Bae (J. Control Release, 153:198-205, 2011) and Waite (Crit. Rev. Biomed. Eng., 40:21-41, 2012).

The biological conundrum for macrophage-dependent or associated diseases is that particles must by adequately small to efficiently extravasate from heterogeneously enhanced vascular permeability, but not too small or they will be rapidly cleared from the blood by the kidney with a resulting inadequate blood life. Outside the capillary, small size facilitates diffusion through the extracellular space, which has little fluid flow and even reduced diffusion within the increased tissue matrix. And then to appreciably change scattering, they must eventually become quite large and adequately concentrated. Thus, the full participation of activated macrophages is required in order to create the enhanced permeability, and then to sequester and aggregate the small particles sufficiently to effectively scatter energy of various wavelengths. For biological safety, it may also be useful to have the nanoparticles composed of an essential element such as iron.

Taken together, these separate observations lead to the surprising finding that administered USPIOs are cleared from their biologic fluids by macrophages, wherein their sequestration and aggregation in lysosomes increases both particle size and volume fraction sufficiently to be visualized with current medical imaging devices (including medical ultrasound or optical coherence devices, or MEUS and MEOC, respectively). The resulting USPIO aggregates increase scattering and absorption of a wide range of probing wavelengths, including those of light, radiofrequency, microwave and ultrasound. The accumulation of USPIOs in the tissue macrophages has great medical utility because of the wide variety of diseases that displace fixed macrophages or are associated with local accumulation of activated macrophages.

With the inventions generally described, the sections below provide further detailed description for the various aspects of the invention, which aspects should be viewed as parts of the invention as a whole.

### 2. USPIOs and Formulations

The general structure of iron oxide nanoparticles (e.g., USPIO nanoparticles) useful for this invention consist of a magnetite/hemagnetite (e.g., the ferrous/ferric) core that is usually less than 10-20 nm in size. Typically, about 5-20 k iron atoms reside within a crystalline core that has a single magnetic domain. The core is typically coated with a biologically compatible polymer (e.g. sucrose, dextran, or other synthetic carbohydrate) that creates a nanoparticle between 10 and 50 nm in size. These USPIO nanoparticles have been developed to serve as contrast agents or biomarkers for MRI, and are widely used for diagnostic applications both in vitro and in vivo, and, in one instance, for the treatment of iron deficiency anemia.

The macrophage-targeting iron oxide nanoparticle of the invention are small enough to slowly escape from the capillaries in or near the macrophage-dependent or -associated disease, yet remains in the circulation for a sufficient time to allow this process to be efficient. Examples of nanoparticles with these characteristics include ferumoxytol and ferumoxtran-10.

In certain embodiment, a coating is additionally added to the nanoparticles to allow safer administration of the iron oxide nanoparticulate parenterally, and to increases the blood half-life of the nanoparticles. The coating may further provide a chemical scaffolding to facilitate loading of the final nanoparticulate with one or more therapeutic agents, thus creating a macrophage-based depot source of therapeutics.

The design of the combination product can be simply as a macrophage-targeted combination, or the design of the combination may include materials that allow increased release of the therapeutic due to, for example, the low pH in the endosome and/or the higher temperature created by local heating.

In essence, either of these capabilities allow for a depot source of therapeutic drug within the macrophage, which depot source may provide slow delivery of the therapeutic to the disease (e.g., inflammatory) site, or the release could be enhanced by local heating or pH change.

There are a large number of USPIO formulations suitable for the instant invention. For example, MRI contrasting agents SPIOs and USPIOs have been approved for commercial use. They include any of the following: Feridex I.V. (also known as Endorem and ferumoxides), Resovist (also known as Cliavist, approved for the European market in 2001), and Feraheme (ferumoxytol). For a review of USPIO formulations, see Corot (Adv. Drug Del. Rev., 58:1471-1504, 2006). As long as the size requirement is met, the USPIO nanoparticles or functionalized derivatives thereof (see below) mentioned in the above literature can all be used in the instant invention.

Change in the quantity or distribution of loaded macrophages may also be used to assess therapeutic response or lack thereof in that subject.

TABLE 1
Selected Parameters of Iron Oxide Nanoparticles

| Attribute | FERIDEX ® | FERAHEME ® | COMBIDEX ® |
|---|---|---|---|
| Size (nm) | 120-180 | 30 | 15-30 (Mean 21) |
| Recommended dose | 0.6 mg/kg | 7 mg/kg | 2.6 mg/kg |
| Blood T_{1/2} | 2 hours | 10-14 hours | 24-36 hours |
| Macrophage | Liver ++++ | Liver +++ | Liver ++ |
| Imaging Utility | Spleen +++ | Lymph node + | Lymph node ++ |
| | LN/IAM 0 | IAM ++ | IAM ++ |

Ferumoxytol is one of the USPIO nanoparticles suitable for assessing local vascularity and macrophage uptake, due to its intermediate pharmacokinetics and the exceptional clinical experience of its use in treating iron-deficient anemia. Ferumoxytol, as a representative USPIO, has potent superparamagnetic properties that make it ideal for use as a MR contrast agent. Due to its slow blood clearance, ferumoxytol remains largely in the vascular space at early time points (minutes to hours after intravenous administration) where it can be used to quantify vascular perfusion and blood volume in regions of interest using appropriate MRI techniques. Ferumoxytol is then removed from the blood entirely by macrophages, where it is aggregated in their lysosomes. USPIOs remain effective as phagocytic biomarkers until the nanoparticle is degraded in macrophages.

The high surface area-to-volume ratios of the nanoparticle yields high drug loading capacities. Thus, in certain embodiments, the nanoparticles of the invention (e.g., the USPIO particles) can be loaded with therapeutic agents that may be concentrated in disease-associated macrophages, thus turning these macrophages into a depot for the therapeutic agents. The therapeutic agents may be gradually released based on, for example, local pH change when the coated nanoparticles enters a low pH environment within the macrophage, such as the endosomal compartment. The therapeutic agent may also be released upon heating the nanoparticles according to the method of the invention.

In certain embodiments, the subject nanoparticles are surface protected and/or functionalized by hydrophilic groups, such as biocompatible polymers. Such hydrophilic “shell” improves the stability in aqueous systems and may extend serum half-life.

In certain embodiments, the subject nanoparticles are surface functionalized by polymeric micellar structures based on amphiphilic block copolymers maleimide-terminated poly-(ethylene glycol)-block-poly(D,L-lactide) copolymer (MAL-PEGPLA) and methoxy-terminated poly(ethylene glycol)-block-poly-(D,L-lactide) copolymer (MPEG-PLA). Such coating can be loaded with chemotherapeutic drugs, such as doxorubicin (DOX). The thiol reacting maleimido terminal group allows such coated nanoparticles to be linked to one or more thiol group-containing peptide or drugs agents via thiol-maleimide reaction. Different extents of protein or drug loading can be achieved through control of the amount of MPEG-PLA introduced into the system. See, for example, Guthi et al. (Mol. Pharmaceutics, 7:32-40, 2010), and Nasongkla et al. (Nano Lett., 6:2427-2430, 2006).

In certain embodiments, the subject nanoparticles are surface functionalized by biodegradable and biocompatible poly(acrylic acid). See, for example, Santra et al. (Small Weinheim an der Bergstrasse, Germany) 5(16):1862-1868, 2009), for the use of biodegradable and biocompatible poly(acrylic acid)-iron oxide nanoparticles (PAAIONPs) functionalized with anticancer drug, Taxol, by a solvent-diffusion method. Drug release was only observed in acidic pH or in the presence of esterase, a degradative enzyme.

In certain embodiments, the subject nanoparticles are surface functionalized by a therapeutic agent (e.g., an anticancer drug, such as methotrexate (MTX) that is linked to the nanoparticle via a peptide linkage that is reducible under acidic pH. Thus upon entry of the nanoparticle into the acid macrophage lysosomal/endosomal compartment, the acid labile linkage is broken, thereby releasing the drug into the cell. See, for example, Kohler et al. (Langmuir 21:8858-8864, 2005).

In certain embodiments, the subject nanoparticles are surface functionalized by N-phosphonomethyl iminodiacetic acid (PMIDA). This amine-functionalization can be performed via a two-step process. First, the USPIOs were synthesized by an alkali-mediated chemical coprecipitation process in the presence of PMIDA containing one phosphonic and two carboxylic groups. This process creates stable nanoparticles with high interparticle repulsion due to negatively charged carboxylic and phosphonate groups on the surface. An amine-functionalized surface is then generated by reacting the terminal carboxylic acids with diamine (EDBE) in the presence of EDC and NHS. This amine functionality can be further utilized to introduce a moiety containing an amine reactive group, such as a drug moiety (e.g., the chemotherapy drug MTX). See, for example, Das et al. (Small Weinheim an der Bergstrasse, Germany) 5:2883-2893, 2009).

In certain embodiments, the subject nanoparticles are surface functionalized by phosphonate groups, or carboxylate groups. Compared to phosphonate groups, bonds formed through the carboxylate groups have decreased thermal stability and increased susceptibility to enzymatic degradation, and may be more suitable for controlled release of drug moieties conjugated to the nanoparticle surface. Das (supra).

In certain embodiments, the subject nanoparticles are surface functionalized by monodisperse, discretemesoporous silicamaterials. Various agents, including therapeutic agents (e.g., doxorubicin or (DOX)), can be encapsulated within the silica matrix. The surface of the nanoparticles may be further functionalized with PEG groups. See, for example, Kim et al. (Angew. Chem., Int. Ed. 47:8438-8441, 2008)

In certain embodiments, the subject nanoparticles are surface functionalized by, for example, a cloaking agent such as poly(ethylene glycol) (PEG).

In certain embodiments, the subject nanoparticles are surface functionalized by targeting ligands that direct the nanoparticles to or near disease-associated macrophages. The targeting ligands may include those that target surface ligands or receptors of disease associated macrophages or diseased cells (such as cancer cells) surrounding the disease associated macrophages. Exemplary targeting ligands may bind to various cell surface receptors, such as transferin receptor (e.g., bound by holo-transferin ligand), folate receptor (e.g., bound by α-folate receptor-targeting folic acid groups), and human/epidermal growth factor receptor 2, upregulated on the surface of various cancer cells. Exemplary targeting ligands may include, without limitation, biotin, avidin, antibody, monoclonal antibody, phage, folate, aptamer, protein or a binding fragment thereof.

Introduction of targeting ligands in general may help to further increase the target-to-background contrast in imaging and improve the local concentration of the therapeutic at the target of interest, with the goal of reducing systemic toxicity. Although this may not be essential where only disease-associated macrophages phagocytize the subject nanoparticles.

In certain embodiments, the subject iron oxide nanoparticles may be functionalized or coated with a biocompatible polymer, such as PEG, PEI, etc., and can be used to deliver a variety of hydrophobic/hydrophilic therapeutic drugs, such as anticancer drugs. Suitable anticancer drugs may include chemotherapeutic agents, including paclitaxel, doxorubicin, methotrexate, and camptothecin.

In certain embodiments, the subject iron oxide nanoparticles may be functionalized with targeting ligands and/or therapeutic reagents that are covalently attached to the dextran-coated monocrystalline iron oxide (Tf-MION), or cross-linked iron oxide (Tf-CFIO). The functionalized nanoparticles can easily be taken up by macrophages via receptor-mediated endocytosis or phagocytosis. Such covalent linking methods have been described in, for example, Ichikawa et al. (“MRI of transgene expression: Correlation to therapeutic gene expression” Neoplasia (New York, N.Y., United States) 4:523-530, 2002); Moore et al. (“Human transferrin receptor gene as a marker gene for MR imaging,” Radiology (Oak Brook, Ill., United States) 221:244-250, 2001); and Weissleder et al. (“In vivo magnetic resonance imaging of transgene expression,” Nat. Med., (New York) 6:351-354, 2000). All are incorporated herein by reference.

In certain embodiments, the subject iron oxide nanoparticles may be functionalized with targeting ligands and/or therapeutic reagents that are covalently attached to or associated with Poly(ethyleneimine) (e.g., branched PEI, MW10 kDa). PEI is positively charged, and may permit negatively charged therapeutic agents to be associated with the nanoparticle. Upon entry of a low pH environment, the therapeutic agent may be protonated and lose its negative charge, thus dissociating from the PEI coating and be released. PEI coating of the nanoparticle is known in the art. See, for example, Park et al. (Biomaterials, 29:724-732, 2008, incorporated herein by reference).

In certain embodiments, the subject iron oxide nanoparticles may be coated by dextran.

In certain embodiments, the subject iron oxide nanoparticles may be coated by linear cyclodextrin-containing polycations (CDPs) for in vivo delivery of nucleic acids, such as DNA-based therapeutic agents (e.g., expression vectors or DNAzyme) or RNA-based therapeutic agents (e.g., siRNA, miRNA, shRNA, ribozyme). See, for example, Hu-Lieskovan et al., (“Sequence-specific knockdown of EWS-FLI1 by targeted, nonviral delivery of small interfering RNA inhibits tumor growth in a murine model of metastatic Ewing's sarcoma,” Cancer Res. 65:8984-8992, 2005), and Pun et al. (“Targeted delivery of RNA-cleaving DNA enzyme (DNAzyme) to tumor tissue by transferrin-modified, cyclodextrin-based particles,” Cancer Biol. Ther., 3:641-650, 2004). Iron oxide nanoparticles so coated may be systemically injected into a subject (e.g., via I.V. injection).

In certain embodiments, the subject iron oxide nanoparticles may be coated by a carrier for a therapeutic agent that releases the therapeutic agent at a low pH environment, such as inside the lysosomal/endosomal compartment of a macrophage. One of such carrier can be calcium phosphate-based delivery vehicles that can be effective for the delivery of hydrophobic, insoluble drugs such as ceramide and the chemotherapeutic agent camptothecin. Calcium phosphate coated nanoparticles are relatively insoluble at physiological pH (pH=7.4) but have improved solubility in acidic pH environments (pH=6.5). Upon endocytosis, calcium phosphate will release the drug only in acidic compartments of the cell (endosomes and lysosomes), thus further reducing the off-target toxicity in systemic administration. This pH-tunable solubility has been used to develop calcium phosphate-based vehicles for controlled, pH-triggered drug delivery. See, for example, Barth et al. (“Bioconjugation of calcium phosphosilicate composite nanoparticles for selective targeting of human breast and pancreatic cancers in vivo,” ACS Nano., 4:1279-1287, 2010).

In certain embodiments, the calcium phosphate nanoparticles may be further functionalized by amine carboxylate- and/or poly(ethylene glycol)-groups. See Kesteret et al. (“Calcium phosphate nanocomposite particles for in vitro imaging and encapsulated chemotherapeutic drug delivery to cancer cells,” Nano. Lett., 8:4116-4121, 2008). Such colloidally stable, amine carboxylate- and poly(ethylene glycol)-functionalized calcium phosphate nanoparticles (CPNPs) can be used for delivery of anti-cancer agent ceramide (Cer6 and Cer10).

In certain embodiments, the CPNP particles may be further functionalized with PEG groups or other protein agents such as antibodies. See, Barth et al. (supra).

In certain embodiments, the subject iron oxide nanoparticles may be coated by poly(ethylene oxide) (PEO)-modified poly(β-amino ester) (PbAE)-based materials, or PEO-poly(caprolactam) (PCL)-based materials, which may be useful as pH-responsive, nontoxic, and biodegradable nanoparticle drug carriers. Solid unprotonated PbAEs are insoluble at physiological pH. However, the solubility increases upon protonation of the amines along the backbone. At below pH 6.5, PbAEs release their payload of chemotherapeutic drug, such as paclitaxel.

### 3. Dose, Administration Route, Treatment Regimen

Macrophages can sequester intravenous doses that are twenty times larger than those required for MRI (usually 0.5-7 mg/kg), and these sequestered particles retain their superparamagnetic properties (indicating persistent nanoparticulate form) for weeks. Preclinical safety studies with a representative USPIO show that doses 200 times the imaging dose can be administered and removed from body fluids by macrophages. See Bourrinet (Invest. Radiol., 41:313-324, 2006). Clearly, the capacity of phagocytosis of USPIOs is very large.

As the USPIO dose increases, the size of nanoparticulate aggregates in macrophage lysosomes increases along with the volume fraction of aggregated nanoparticles. The effective amount/dose of USPIO, the route of administration, and the delay time for selective accumulation in macrophages can all be used to control or optimize the imaging and/or treatment process. It is the selective nature of aggregation in tissue macrophages that provides a utility of the invention.

The intravenous dose for MRI can vary based in part on the route of administration, the magnetic field strength, and the structures that need to be separately visualized. Table 1 shows the most common doses for MRI utility. However, macrophage uptake depends upon both the presenting fluid for the macrophages, the time to accumulate and then metabolize the ingested USPIOs, and the purpose of the administration—whether for diagnosis or therapy.

For intravenous loading of TAMs or IAMs, the following regimens may be used: a dose of about 0.5 to 20 mg/kg, about 1 to 10 mg/kg, or about 2 to 5 mg/kg.

In certain embodiments, the lower limit of the dose may be about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.2 mg/kg, about 1.5 mg/kg, about 2.0 mg/kg, or about 2.5 mg/kg.

In certain embodiments, the upper limit of the dose may be about 100 mg/kg, about 50 mg/kg, about 30 mg/kg, about 20 mg/kg, about 18 mg/kg, about 16 mg/kg, about 15 mg/kg, about 14 mg/kg, about 12 mg/kg, about 10 mg/kg, about 9 mg/kg, about 8 mg/kg, about 7 mg/kg, about 6 mg/kg, or about 5 mg/kg.

In certain embodiments, the lower limit of the dose is chosen from any one of: about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.2 mg/kg, about 1.5 mg/kg, about 2.0 mg/kg, or about 2.5 mg/kg, and the upper limit is chosen from any one of about 100 mg/kg, about 50 mg/kg, about 30 mg/kg, about 20 mg/kg, about 18 mg/kg, about 16 mg/kg, about 15 mg/kg, about 14 mg/kg, about 12 mg/kg, about 10 mg/kg, about 9 mg/kg, about 8 mg/kg, about 7 mg/kg, about 6 mg/kg, or about 5 mg/kg.

In certain embodiments, any of the doses may be repeated as necessary. The repeat dose may be the same or different from the previous dose. In certain embodiments, each repeat has about the same dose.

Optionally, the waiting time between the repeats may be between 12 (0.5 day) and 144 hours (12 days) or longer, or between 1-10 days, or between 2-10 days, between 3-10 days, between 4-10 days, between 5-10 days.

In certain embodiments, the waiting time between the repeats may be at least about 1, 2, 3, 4, 5, 6, or 7 days.

In certain embodiments, the waiting time between the repeats may be up to 20, 18, 16, 15, 14, 12, 10, 9, 8, 7, 6, or 5 days.

In certain embodiments, the USPIO nanoparticles or formulations thereof may be administered interstitially, e.g., for nodal enhancement. Suitable doses for this administration route may be a dose of about 0.01-2 mg/kg, about 0.02-1.5 mg/kg, about 0.03-1 mg/kg, about 0.05-1 mg/kg, about 0.1-1 mg/kg, about 0.2-0.5 mg/kg.

In certain embodiments, the lower range of the dose may be any one of: about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.2 mg/kg, or about 1.5 mg/kg.

In certain embodiments, the upper limit of the dose may be about 10 mg/kg, about 8 mg/kg, about 6 mg/kg, about 4 mg/kg, about 2 mg/kg, about 1.9 mg/kg, about 1.8 mg/kg, about 1.6 mg/kg, about 1.5 mg/kg, about 1.4 mg/kg, about 1.2 mg/kg, or about 1.0 mg/kg.

In certain embodiments, the lower limit of the wait time is about 10 min, 20 min, 30 min, 40 min, 50 min, 1 hr, 1.5 hr, 2 hr, 3 hr, 6 hr, 12 hr, 18 hr, 1 day, 2 days, 3 days, 5 days, or 7 days, and the upper limit of the wait time is about 20 days, 18 days, 16 days, 15 days, 14 days, 13 days, 12 days, 11 days, 10 days, 9 days, or 8 days.

In certain embodiments, the administration is carried out with stimulation of lymphatic uptake as feasible.

The administration may be repeated as necessary, with wait time between repeats between 30 min to 14 days.

In certain embodiments, the USPIO nanoparticles or formulations are administered intracavitarily. In certain embodiments, the lower limit of the wait time is about 10 min, 20 min, 30 min, 40 min, 50 min, 1 hr, 1.5 hr, 2 hr, 3 hr, 6 hr, 12 hr, 18 hr, 1 day, 2 days, 3 days, 5 days, or 7 days.

In certain embodiments, the upper limit of the wait time is about 20 days, 18 days, 16 days, 15 days, 14 days, 13 days, 12 days, 11 days, 10 days, 9 days, or 8 days.

In certain embodiments, the lower limit of the wait time is about 10 min, 20 min, 30 min, 40 min, 50 min, 1 hr, 1.5 hr, 2 hr, 3 hr, 6 hr, 12 hr, 18 hr, 1 day, 2 days, 3 days, 5 days, or 7 days, and the upper limit of the wait time is about 20 days, 18 days, 16 days, 15 days, 14 days, 13 days, 12 days, 11 days, 10 days, 9 days, or 8 days.

Suitable dose includes 0.05-2 mg/kg, preferably in an appropriate suspension. The pre-determined wait time is about 30 min-14 days before imaging.

In certain embodiments, the lower limit of the dose is about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.2 mg/kg, about 1.4 mg/kg, or about 1.5 mg/kg.

In certain embodiments, the upper limit of the dose is about 10 mg/kg, about 8 mg/kg, about 6 mg/kg, about 5 mg/kg, about 4 mg/kg, about 3 mg/kg, about 2 mg/kg, about 1.9 mg/kg, about 1.8 mg/kg, about 1.7 mg/kg, about 1.6 mg/kg, about 1.5 mg/kg, about 1.4 mg/kg, about 1.3 mg/kg, about 1.2 mg/kg, about 1.1 mg/kg, or about 1.0 mg/kg.

In certain embodiments, the lower limit of the pre-determined wait time is about 10 min, 20 min, 30 min, 40 min, 50 min, 1 hr, 1.5 hr, 2 hr, 3 hr, 6 hr, 12 hr, 18 hr, 1 day, 2 days, 3 days, 5 days, or 7 days.

In certain embodiments, the upper limit of the pre-determined wait time is about 20 days, 18 days, 16 days, 15 days, 14 days, 13 days, 12 days, 11 days, 10 days, 9 days, or 8 days.

In certain embodiments, the lower limit of the pre-determined wait time is about 10 min, 20 min, 30 min, 40 min, 50 min, 1 hr, 1.5 hr, 2 hr, 3 hr, 6 hr, 12 hr, 18 hr, 1 day, 2 days, 3 days, 5 days, or 7 days, and the pre-determined upper limit of the wait time is about 20 days, 18 days, 16 days, 15 days, 14 days, 13 days, 12 days, 11 days, 10 days, 9 days, or 8 days.

### 4. Heating of Aggregates

The USPIO aggregates within the activated macrophages can be selectively heated by a variety of energy sources, such as light (laser), RF, microwave, and ultrasound. There are no effective barriers to the transmission of the nanoparticle heat within the macrophage and thence to immediately adjacent structures, including other pathologic targets. Depending upon the degree and duration of the heating, thermotherapy or thermoablation of the heated cells or thermorelease of drug can be accomplished.

The intermediate wavelengths that characterize radiofrequency and microwave have already been used for heating tissues. Indeed, all of these wavelengths can be associated with scattering and energy absorption by USPIO nanoparticles, and the efficiency of their size follows the scattering theories listed herein (Rayleigh and Mie scattering). An additional factor to consider is the volume fraction of the tissue region containing aggregated nanoparticles.

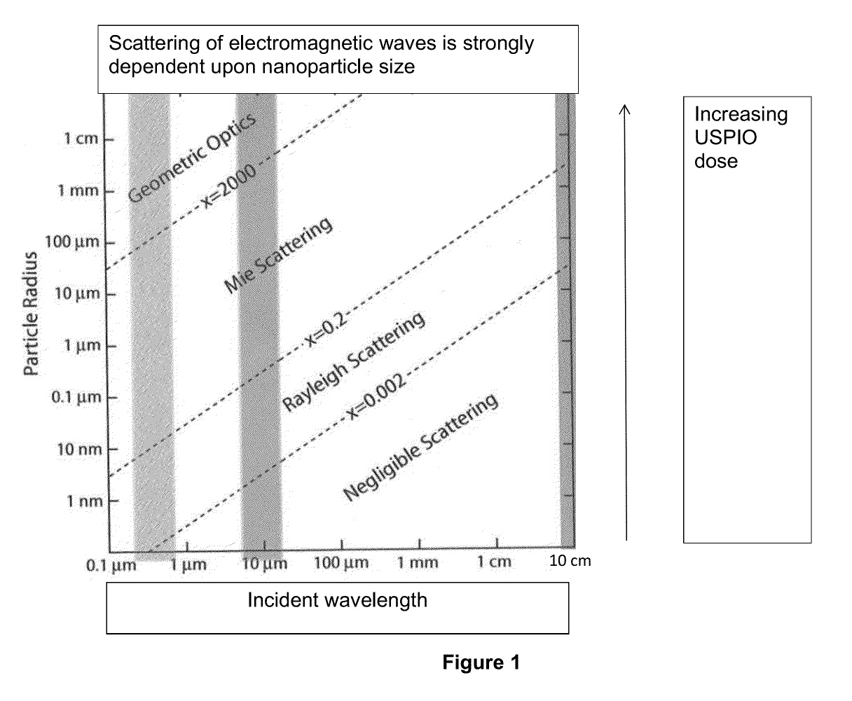

In the graphic solution of the two scattering domains shown in FIG. 1, the relation between nanoparticle size and probing wavelength is based upon Rayleigh and Mie scattering. The values for x are the ratios between particle size and wavelength. The illustration also shows that many common particulates scatter electromagnetic waves in proportion to their size. This is true across the electromagnetic spectrum. In as much as all tissues can be damaged or killed by sustained heating, selective heating of the target tissue is desirable.

The scattering of impinging electromagnetic waves is followed by absorption of the delivered energy, in turn leading to increase in temperature near the location where this absorption occurs. This heat is then conducted to surrounding tissues through thermal conduction. When the local temperature reaches certain value, cell damage begins in proportion to the increase in temperature and its duration.

Serving as a heat sink, the activated macrophages having the large USPIO aggregates essentially become the target for hyperthermic ablation. Cells heated above 42° C. begin to show signs of apoptosis. Temperatures above 50° C. are less associated with apoptosis and more with frank necrosis. These outcomes are time and temperature dependent.

The USPIO heating is particularly effective when the particles are targeted so that the heating is selective for the target tissue. For USPIOs, the incremental heating could be accomplished by increasing the effective particle size and/or their fractional volume within the targeted tissue macrophages. In activated macrophages, increasing the size of the lysosomal USPIO aggregates increases the scattering efficiency, thus producing an improved selective heating (and may be killing) of the cells in the surrounding tissue.

In certain embodiments, the size of the aggregates or the loading of the macrophages is controlled by the dose and the time these particles are available in the blood, tissue fluid or lumen. The regimens offered for imaging may be appropriate for thermal therapy as well, but the energy source selected for the particular heating regimen is influenced by the local nanoparticle size that is best suited for the thermal therapy.

FIG. 1 lists the effective particle sizes for Rayleigh and Mie scattering, and it is clear that lower macrophage USPIO uptake is required for laser devices operating in the ultraviolet, visible, and thermal infrared wavelengths. Any laser system designed for photodynamic therapy in that anatomic region will be able to efficiently heat USPIO-loaded macrophages in that region.

Microwave are radiofrequency devices, and are limited by ISM bands which restrict frequency of use in hospitals to designated frequencies—microwave (ISM bands of 433, 975, or 2450 MHz), radiofrequency (ISM bands of 13.5, 27, and 40 GHz). Any radiofrequency or microwave system that is designed to produce hyperthermia by frictional stimulation of water will also be more efficient when heating USPIO enhanced macrophages in the same region.

Ultrasound energy absorption with nanoparticles must be experimentally measured as described in the examples, but imaging frequencies range from 2-60 MHz where higher frequencies have less penetration depth. Therapeutic ultrasound will have the same depth limitations, but will require less power than used for high intensity focused ultrasound with USPIOs.

A strength of USPIO theranosis is that the USPIO content at the proposed treatment site can be estimated from imaging studies, and the location and access for the heating devices combined with their heating efficiency for the existing macrophage loading can be derived from the imaging information, and details about the efficiency of heating for a given hyperthermia plan. The duration of heating and the power applied to the heating device are controllable—the resulting local temperatures can be determined during treatment via probes or MR thermal imaging, and eventually biothermal simulations will provide useful treatment plans. Such simulations have evolved for hyperthermia with high intensity focused ultrasound. See Chopra (Int. J. Hyperthermia, 26:804, 2010). The examples include experiments that can measure conversion of electromagnetic energy into heat when the anatomic region includes UPSIO-loaded macrophages.

The capacity of body macrophages to sequester USPIOs appears to be quite large. Ferumoxytol doses of 1 gram do not appear to alter blood clearance by this widely distributed cell population. Indeed, USPIOs appear to occupy only two body compartments—initially the blood following intravenous administration, and then the macrophages. Some macrophages such as those in the liver or spleen can ingest the USPIOs directly from blood. Due to their size, in other tissues they leak from the blood very slowly. Upon exit from the blood in these tissues, the nanoparticles are quickly phagocytized by local macrophages where they appear to remain until metabolized. These same macrophages, in disease states, facilitate the leakage of the USPIOs through angiogenic cytokines. Many important disease states contain activated macrophages within responding tissues. These macrophages can be related to cancer (tumor-associated macrophages, TAMs) or any macrophage-dependent inflammatory process (inflammation-associated macrophages or IAMs).

When it is desirable to selectively ablate tissue containing macrophages by heating regimens, these macrophages can be loaded with USPIO nanoparticles whose size and volume fraction are increased by the lysosomal aggregation of the administered USPIOs within the local macrophages.

The amount and distribution of the administered UPSIOs can be determined with imaging devices sensitive to the altered electromagnetic scattering or change in local tissue relaxivity. The duration of effective amounts of phagocytosed USPIOs is dependent upon their metabolism, but may be sustained for a period of days and weeks—this allows repeated hyperthermic dosing over time or in multiple macrophage-rich body regions. This would also facilitate the co-administration of chemotherapy or radiotherapy.

Delivering the effective amount of electromagnetic energy to achieve the desired increase in local temperature and its duration will depend upon the hearing device used and the local USPIO content, but in every case the efficiency of local tissue heating near the USPIO-containing macrophages will be enhanced. A wide variety of electromagnetic wavelength could be used to heat the USPIO-loaded macrophages, including light (laser), RF, microwave, and ultrasound. This process provides a selective local heat sink. Due to their proximity to the inflammatory pathophysiology of interest, the heat absorbed by the USPIO-loaded macrophages is thermally conducted to the targeted tissues. At times, the necessary destruction of the loaded macrophages is desirable as they may be sustaining the inflammatory disease process.

In certain embodiments, the USPIO nanoparticles are coated with a carrier that contains therapeutic drugs, and heating of the nanoparticles facilitates the release of the therapeutic drugs bound to the nanoparticles. This local release from the co-delivered nanoparticle will create a much higher concentration of the therapeutic drug, thus creating a favorable therapeutic ratio versus other tissues that might limit the parenteral dosing with the agent absent this local targeting.

### 5. Diseases, Disorders, or Conditions

Although the inflammatory disease process may be widely distributed, the uptake of USPIOs is very focal. Indeed, many inflammatory diseases that create significant morbidity are focal in their expression; e.g., atherosclerosis creating vulnerable plaque is limited to local arterial sites, although there may be many such sites. The focal nature of the activated macrophage accumulation of USPIOs provides exceptional opportunities for imaging and selectively treating these diseases. Other examples of the highly focal diseases of interest include inflammatory bowel diseases (IBD) and primary cancers. Even when cancer disseminates, the cancer cells are all close to a capillary, and, if aggressive, intimately enmeshed with tumor-associated macrophages. Thus, in macrophage-dependent or -associated diseases, the active process is always adjacent to a capillary, the increased capillary permeability provides a source of USPIOs from blood, and the activated macrophages accumulate virtually the entire local nanoparticle load. One basis of the invention is that the USPIOs accumulated in activated macrophages are not only markers of the disease, but can also be utilized to both identify the location and extent of disease and, surprisingly, to treat the disease focally with theranostic devices combining detection and treatment.

In certain embodiments, the macrophage-associated disease or condition is an “inflammatory disease, disorder, or otherwise abnormal condition,” which may include disorders associated with inflammation or have an inflammation component, such as, but are not limited to: acne vulgaris, asthma, COPD, autoimmune diseases, celiac disease, chronic (plaque) prostatitis, glomerulonephritis, hypersensitivities, inflammatory bowel diseases (IBD, Crohn's disease, ulcerative colitis), pelvic inflammatory disease, reperfusion injury, rheumatoid arthritis, sarcoidosis, transplant rejection, vasculitis, interstitial cystitis, atherosclerosis, allergies (type 1, 2, and 3 hypersensitivity, hay fever), inflammatory myopathies, as systemic sclerosis, and include dermatomyositis, polymyositis, inclusion body myositis, Chediak-Higashi syndrome, chronic granulomatous disease, Vitamin A deficiency, cancer (solid tumor, gallbladder carcinoma), periodontitis, Granulomatous inflammation (tuberculosis, leprosy, sarcoidosis, and syphilis), fibrinous inflammation, purulent inflammation, serous inflammation, ulcerative inflammation, and ischaemic heart disease, type I diabetes, and diabetic nephropathy.

In certain embodiments, the inflammatory disease, disorder, or otherwise abnormal condition includes many autoimmune diseases or disorders that are associated with inflammation or have an inflammation component, e.g., corresponding to one or more types of hypersensitivity. Exemplary autoimmune diseases or disorders that correspond to one or more types of hypersensitivity include: atopic allergy, atopic dermatitis, autoimmune hemolytic anemia, autoimmune hepatitis, autoimmune polyendocrine syndrome, autoimmune urticaria, celiac disease, cold agglutinin disease, contact dermatitis, Crohn's disease, diabetes mellitus type 1, discoid lupus erythematosus, Erythroblastosis fetalis, Goodpasture's syndrome, Graves' disease, Guillain-Barré syndrome (GBS), Hashimoto's encephalopathy, Hashimoto's thyroiditis, idiopathic thrombocytopenic purpura, autoimmune thrombocytopenic purpura, IgA nephropathy, lupus erythematosus, Ménière's disease, multiple sclerosis, myasthenia gravis, narcolepsy, neuromyelitis optica, Devic's disease, neuromyotonia, ocular cicatricial pemphigoid, opsoclonus myoclonus syndrome, PANDAS (pediatric autoimmune neuropsychiatric disorders associated with streptococcus), paraneoplastic cerebellar degeneration, pemphigus vulgaris, pernicious anaemia, psoriasis, psoriatic arthritis, rheumatoid arthritis, rheumatic fever, sarcoidosis, scleroderma, subacute bacterial endocarditis (SBE), systemic lupus erythematosis, Lupus erythematosis, temporal arteritis (also known as “giant cell arteritis”), thrombocytopenia, ulcerative colitis, undifferentiated connective tissue disease, urticarial vasculitis, and vasculitis.

This section further describes several representative diseases, disorders, or conditions that can be imaged and/or treated with the methods of the invention. The diseases, disorders, or conditions listed herein are for illustrative purpose only, and are not limiting.

### Breast Cancer

There are multiple utilities for USPIO identification of macrophages in the staging and management of breast cancer, as well as responses to treatment, as set for below.

A) Following the clinical diagnosis of breast cancer, a staging MEMRI (macrophage enhanced MRI) is performed (e.g., according to the method of US 2009-0004113, incorporated by reference) to capture vascular information in the early distribution following intravenous administration, and the macrophage information after an appropriate delay (e.g., primary and metastatic tumor-associated macrophages, macrophage displacement in liver, lymph node, bone marrow, or adrenal cortex, see US 2012/0003160, incorporated by reference). The MR imaging methods are familiar to those skilled in the art. At this time, it may be desirable to evaluate regions for USPIO-enhanced macrophages with ultrasound to determine if there is sufficient loading to utilize ultrasound for further evaluation of the clinical stage. Search for macrophage-enhanced metastasis can be performed as indicated. The intent is to provide complete imaging TNM (Tumor, Node, Metastasis) staging.

B) Prior to surgery, percutaneous lymphography can be performed (see, e.g., U.S. Pat. No. 5,496,536, incorporated herein by reference). Here the USPIO administration site, dose and time are selected based upon whether sentinel node status will be evaluated with MRI or ultrasound. Steps A) and B) can be combined as best suited for the particular clinical circumstance.

C) Surgical evaluation of the sentinel node. The location of the sentinel node(s) and other nodes of interest is determined in step A) and/or step B). These nodes can be removed prior to definitive care of the primary breast cancer or at the same time as the primary cancer is removed or biopsied. The presence of USPIO in the lymph nodes may be directly visible due to its dark coloration and interactive ultrasound evaluations can be included in the sentinel node removal.

D) Tissue diagnosis of the primary tumor. If it is desirable to obtain a histologic evaluation of the primary tumor when tumor-associated macrophages are visualized with either MRI or ultrasound, either of these modalities can be used to direct biopsy.

E) Surgical Excision of the primary tumor. Using the MEMRI (macrophage enhanced MRI) or MEUS (macrophage enhanced ultrasound) information, the surgical approach to the primary tumor can be planned. It may be useful to correlate a contemporary MEMRI with breast ultrasound prior to surgery. If the TAMs (Tumor-Associated Macrophages) are ultrasonically evident, then ultrasound can be used during the surgical extirpation to guide confirmation of margins and successful removal of all tumor and the associated TAMs. This can be confirmed with a post-op imaging procedure, preferably MEMRI.

F) Monitoring adjuvant or supplemental chemotherapy or radiation therapy. MEMRI or MEUS can be used as indicated subsequently to evaluate the therapeutic response to treatment of primary or metastatic breast cancer.

G) USPIO thermotherapy, chemothermotherapy, chemotherapy, or adjuvant radiation and/or systemic chemotherapy. Care of the subject is personalized with the use of the USPIO-enhanced images and with the option to use either USPIO, USPIO thermal therapy, USPIO chemotherapy, or the best combinations.

H) At appropriate points in the care of the patient, the aggressiveness of primary or metastatic cancer may be evident on MEMRI or MRUS, since the activity of TAMs is an indicator of the aggressiveness of that cancer in the this patient. This information can be used to estimate prognosis.

I) USPIO technologies facilitates repeated evaluations throughout the course of the patient's breast cancer care. The information can be used to change any therapy, to continue therapy, or to stop therapy.

Clearly, all of these steps reveal the behavior of the cancer in the afflicted patient and provide the opportunity for truly personalized care of that patient with respect to her breast cancer. Each of the steps can be used individually or in combination as the current stage of the disease and its response to treatment evolve.

Other Cancers with Macrophage-Dependent Components

The methods described above can be adopted as clinically desirable for a wide variety of other cancers. The absence or low level of macrophage involvement may indicate a less aggressive cancer such as ductal carcinoma in situ (DCIS) or low grade prostatic cancer where gentle treatments or even watchful waiting is appropriate.

However, there are many cancers with aggressive TAM (tumor associated macrophages) components where this invention is useful. Glioblastoma, GU, GI, lung, sarcoma, thyroid, and salivary gland tumors are a few that frequently are aggressive and associated with prominent TAMs. See Heusinkveid and van der Burg (J. Translat. Med. 9:216-240, 2011). The teaching of this invention can be used to determine which cancers could usefully have USPIO directed theranosis.

### Macrophage-Dependent Inflammatory Diseases

1) Vulnerable Plaque

a. Imaging USPIO-Enhanced Plaques

Using an acceptable model of vulnerable plaque such as the rabbit or pig, the accumulation of detectable USPIO-loaded macrophages may be assessed with catheter-based imaging techniques such as VH-US or optical coherence tomography (OCT).

Macrophage loading can be varied using post injection time and dose as appropriate. The VH-US and OCT imaging devices are used for detection of the loaded macrophages in the plaque. The accuracy and sensitivity of the imaging performance is compared with vascular histopathology.

b. Treating USPIO-Enhanced Plaques
(i). All of the therapeutic methods elucidated above can be adopted for treatment of the vulnerable plaque with its macrophage-enhanced UPSIO content.
(ii) Focal heating of the loaded macrophages in the vulnerable plaque, energy delivery would usually be from ultrasound or laser devices.
(iii) Positioning of the energy delivery device can be by use of a companion imaging system or from landmarks established from the imaging data. In some circumstances, the difference in energy absorption between USPIO-enhanced areas and other tissues may be large enough that the energy delivery and time at each treatment position can be a simple matter of controlling the pullback positions.
(iv) USPIO chemotherapy

Targeting the macrophages in the vulnerable plaque allows for delivery of composite drugs to those cells.
(v) USPIO chemothermotherapy

In a manner similar to that already described for treatment of cancer foci, the treatment of vulnerable plaque may include use of USPIO condition-dependent formulations where thermal release of the therapeutic moiety is facilitated by combining local heating with the previously administered USPIO plus drug formulation.

2) Inflammatory Bowel Disease (IBD)

Using established animal models of Crohn's disease or ulcerative colitis, MRI, endoscopic ultrasound or endoscopic OCT can be performed and the ability to localize the macrophages as well as detection sensitivity and specificity can be determined in relation to histologic confirmation.

A number of devices are available to perform heat ablation of the colonic mucosa. Among these are deployable RF devices such as the Halo™. There are also established techniques for isolating a segment of colon between two balloons and creating an environment where high intensity ultrasound or optical coherence energy delivery devices can be employed to utilize the loaded macrophages within the affected colonic segment for thermal therapy.

It is apparent that focal thermal therapy, focal drug therapy, and focal chemothermotherapy may also be considered in the therapeutic plan. The range of drugs that suppress the adverse chemokines from activated macrophages is broad and can include anti-inflammatories or genetic manipulations such as siRNA to silence the production of chemokines such as TNFα or VEGF.

3) Periodontal Disease

When the local macrophages are unable to reverse the pathology leading to periodontal disease, they become part of the ongoing destruction. The methods described herein can be used for theranosis. For USPIO imaging, both ultrasound and optical devices are feasible to relate macrophage activity to gum scores. Heating probes or ultrasound/optical delivery devices included in mouthpieces similar to those used for teeth whitening can be developed to take advantage of theranosis with USPIOs.

4) Focal Macrophage-Dependent Inflammatory Joint Diseases

Theranosis for these conditions is similar to that for periodontal disease. The participation of activated macrophages in the affected joint is determined with imaging. Focal RF, microwave, ultrasound, or optical energy delivery could be used to achieve the desired thermal therapy or thermal ablation. Focal chemotherapy from drug carrying USPIOs or combined with focal chemothermal therapy are options expected to become feasible with suitable USPIO regimens.

Those skilled in the art will be able to adopt USPIO theranosis to other macrophage-dependent inflammatory diseases, such as COPD, bronchial asthma, psoriasis, transplant rejection and vascular access stenosis.

### 6. Imaging Devices

Ultrasmall superparamagnetic iron oxide particles (USPIOs, size 10-50 nm) have been developed to serve as contrast agents or biomarkers for MRI. Applicants realized that the introduction of such particles into tissue affects the transmission of many wavelengths from the electromagnetic spectrum, which in turn can be used in medical applications such as imaging or even therapy. In as much as the useful USPIOs are small nanoparticles, they fit the domain of particle scattering as described by the Rayleigh or Mie theories. Thus the imaging devices suitable for practicing the methods of the invention include those that utilize electromagnetic wavelengths that are responsive to Rayleigh or Mie scattering.

### Rayleigh Scattering

Rayleigh scattering is named after the British physicist Lord Rayleigh. It is the elastic scattering of light or other electromagnetic radiation by particles much smaller than the wavelength of the light (e.g., individual atoms or molecules). Rayleigh scattering results from the electric polarizability of the particles, in that the oscillating electric field of a light wave acts on the charges within a particle, thus causing them to move at the same frequency. The particle therefore becomes a small radiating dipole whose radiation is seen as scattered light.

The size of a scattering particle is parameterized by the ratio x of its characteristic dimension r and wavelength λ:

$$x = \frac{2 π r}{λ} .$$

Rayleigh scattering can be defined as scattering in the small size parameter regime x<<1 (e.g., 0.002<x<0.2). Scattering from larger spherical particles is explained by the Mie theory (see below) for an arbitrary size parameter x. For small x the Mie theory reduces to the Rayleigh approximation.

The amount of Rayleigh scattering that occurs for a beam of light depends upon the size of the particles and the wavelength of the light. Specifically, the intensity of the scattered light varies as the sixth power of the particle size (r), and varies inversely with the fourth power of the wavelength (λ). In cells, the volume fraction of the particle population also affects scattering.

Rayleigh scattering is a good approximation of the manner in which light scattering occurs within various media for which scattering particles have a small size parameter. Rayleigh scattering may happen, for example, when light travels through transparent solids and liquids, but is most prominently seen in gases. The strong wavelength dependence of the scattering (˜λ⁻⁴) means that shorter (blue) wavelengths are scattered more strongly than longer (red) wavelengths. As a result, Rayleigh scattering of sunlight in the atmosphere is what makes the sky appear blue.

### Mie Scattering

Named after German physicist Gustav Mie, the Mie solution to Maxwell's equations is also known as the Lorenz-Mie solution, the Lorenz-Mie-Debye solution, or Mie scattering. It describes the scattering of electromagnetic radiation by a sphere. Mie scattering occurs when particles are about the same size as the wavelengths being scattered.

For example, in the atmosphere, dust, pollen, smoke and microscopic water droplets are common causes of Mie scattering which tends to affect longer wavelengths. Mie scattering occurs mostly in the lower portions of the atmosphere where larger particles are more abundant, and dominates when cloud conditions are overcast.

Thus scattering by particles similar to or larger than the wavelength of light is typically treated by the Mie theory, the discrete dipole approximation and other computational techniques.

### Suitable Ultrasonic and Optic Medical Imaging Devices

USPIO aggregation in macrophages allows the visualization of the aggregates with ultrasonic and optic medical imaging devices, operating at wavelengths that cause and detect Rayleigh and Mie scattering by the aggregates. The aggregation in macrophages may also affect acoustic or optical mismatches which influence imaging.

There are numerous ultrasonic and optic medical imaging devices suitable to carry out the method of the invention.

The invention also provides theranostic devices constructed to detect the aggregated nanoparticles with scattered ultrasound or light, and then interactively or sequentially heat the nanoparticles locally with a proper energy source, such as radiofrequency, high intensity ultrasound, or lasers.

Thus in one aspect, the invention provides a theranosis device comprising an imaging device that cause and detects Rayleigh and Mie scattering from the USPIO aggregates, and an energy source that emits an energy suitable to raise the temperature of the USPIO aggregates. The device of the invention may a) visualize USPIO-loaded, activated macrophages in macrophage-dependent disease, and, b) locally heat the disease-responsive macrophages with absorbed energy from radiofrequency, high intensity ultrasound, or laser sources.

In certain embodiment, both the imaging and heating capabilities are contained on the same device/probe.

In certain embodiment, the device of the invention is designed for intracavitary, intravascular or interstitial insertion in order to improve proximity to the inflamed site.

In certain embodiment, the imaging and therapy utilities of the device are used simultaneous or sequentially to accomplish controlled thermal ablation of the focal process containing the identified macrophages.

In certain embodiments, the device may comprise additional elements, or be used with such additional elements, such as those that couple the device to the tissue and/or monitor local tissue temperature.

Suitable sheaths may be required to couple the device to the tissue containing the macrophage-loaded targets. Locally targeted thermotherapy is achieved by enhanced heating of the aggregated nanoparticles within the activated macrophages, creating heat sinks that lead to ablation of tissues within the effectively heated region. The region containing the enhanced macrophages may have an irregular shape, and heating the aggregated USPIO particles allows the therapy to geometrically fit the focal disease.

In some embodiments, thermal sensing technology may be useful.

The invention contemplates combinations selecting one element from each of the following categories:

| Macrophage Detection | Heat delivery | Access for Device |
|---|---|---|
| Ultrasound | Focused Ultrasound | Endoscope |
| Optical coherence | Radiofrequency | Catheter |
| | Microwave | Trocar |
| | Laser | |

Scattering and absorption of the wavelength utilized for either imaging or heating is increased by nanoparticle aggregation in the macrophages, and the amount of scattering and absorption is influenced by the effective aggregated nanoparticle sizes and volume fraction in the enhanced macrophages. Thus, the sensitivity for image detection and for heating will depend upon the device combination selected. Similarly, penetration distances and energy delivered per photon absorbed will depend upon the devices used for this invention.

The various combinations of the imaging device and energy sources of the theranosis device and uses thereof are described below, with illustrative examples for treating specific diseases.

For example, for breast cancer detection and treatment, the presence and location of an aggressive breast cancer incorporating tumor-associated macrophages is first identified with the administration of an effective intravenous dose of USPIO, and imaging the enhanced tumor-associated macrophages with MRI or ultrasound. These images provide information about the next steps, which may include proceeding directly to the USPIO-based therapies or may suggest the additional administration of USPIO to achieve the desired local USPIO content. With suitable conditions, it might be decided to use a device combining ultrasound imaging, high intensity ultrasound for heating and a suitable trocar for positioning the heating element at one or more effective locations within the region to be treated. Temperature monitoring may be desirable unless biothermal simulation is sufficient. When embarking upon focal therapy, the theranostic device may further comprise a probe (same or different from the imagine probe) with high intensity ultrasound treatment capability. The ultrasound imaging frequency may be chosen for best resolution of the size of the enhanced breast cancer, and the ultrasound heating sources has frequencies suitable for the penetration distances required and may include treating one or more zones fitting the region to be heated.

In certain embodiments, under external ultrasound guidance, a trocar is advanced into the region containing the tumor-associated macrophages. The theranogstic device is then advanced and the enhanced TAMs within the effective heating zone of the high intensity ultrasound field is confirmed by the interstitial ultrasound imaging capability. Heating is then delivered to that zone. Where necessary, the theranostic probe can then be repositioned to continue therapeutic heating.

For treating breast cancer, radiofrequency and microwave probes, such those radiofrequency ablation systems marketed under the brand name STARBURST® (AngioDynamics, Latham, N.Y.), or microwave heating devices such as the microwave tissue ablation (MTA) system marketed under the brand name ACCULIS® (Microsulis Medical Limited, Hampshire, UK), or similar devices may be selected, and the features of imaging guidance and temperature monitoring may be desirable.

Similar devices may be used to treat other macrophage associated inflammatory disease for focal theranosis treatment. These may include, but are not limited to, interstitial thermal therapy for primary or metastatic brain cancer, lung cancer, renal cancer, pancreatic cancer, prostate cancer, sarcoma as well as metastases associated with melanoma, breast, prostate. Similarly, focal inflammatory disease such as rheumatoid arthritis of large joints may be approached with this kind of interstitial theranogstic management.

The device of the invention can also be used to treat ulcerative colitis of the rectosigmoid colon. The distribution of the ulcerative colitis in the rectosigmoid region is first imaged to confirm suitability for theranostic intervention. A suitable endoscope is advanced into the inflamed region and the theranostic device is deployed through side channels in the endoscope. In this example, the imaging of the USPIO-enhanced macrophages may be accomplished by endoscopic ultrasound and the thermal therapy is performed using a radiofrequency probe. The ultrasound confirms that that the USPIO distribution within the inflamed colon is within the heating region for the radiofrequency probe. Following application of the thermal therapy for that region, the device and endoscope are repositioned for another thermal application. In this manner, the entire inflamed colon is locally treated.

For this clinical scenario, many endoscopically-guided theranostic combinations are feasible: endoscopic ultrasound plus NIR laser; endoscopic optical coherence tomography plus NIR laser, and HIFU with a suitable sheath as described by Chopak.

The theranosis device of the invention is further suitable for treating intracavitary macrophage-associated diseases accessed via endoscopy, which include but are not limited to cancers of the throat, larynx, esophagus, stomach, duodenum, colon and anus, bronchus, etc., and inflammations of the bronchus, GI tract, joints, etc.

For example, the device of the invention can be used to treat vulnerable plaque of the coronary artery. When coronary catheterization is indicated for identification and possible treatment of one or more vulnerable plaques, virtual histology ultrasound as well as high intensity ultrasound thermal therapy can be performed with a single theranostic probe advanced through the proximal catheter. This probe may consist of one ultrasonic imaging region and one or more high intensity ultrasound sources. As the VH-US probe is withdrawn, each region containing a suitable USPIO-enhanced vulnerable plaque is identified and then heated. Each targeted USPIO enhanced coronary plaque is treated sequentially, as clinically indicated.

Similar devices may be deployed to treat other anatomic regions with vulnerable plaque in like manner. Suitable lesions in the carotid, aorta, renal artery, and peripheral arteries may be subject to effective intravascular thermal therapy.

### 7. Exemplary Utility

The methods and systems of the invention provide useful information (e.g., medical imaging) about the presence or absence of diseases in the interrogated tissues, as well as the specific location, shape and size of the affected tissues. Such information in turn provides basis for further medical analysis, such as disease diagnosis, staging (e.g., disease severity and extent), prognosis, treatment options, the response of the disease to a current treatment regimen, or the recurrence of the disease, etc.

For example, although the macrophage-dependent inflammatory changes are similar in general, cytokine and other cellular responses can differ among individuals or with particular inflammatory diseases. These differences can be in the nature of the disease or in the nature of the response to the disease. Such considerations have led to the postulation of the need for personalized medicine, i.e., treating a particular disease in a particular patient by a regimen that is tailored to that diseased patient. For instance, identifying the actual genetic aberration in a patient's cancer and treating that abnormality with a treatment uniquely successful for the identified abnormality is one form of personalized medicine. However, personalized medicine might encompass assessing the pathophysiology that accompanies a disease process in an individual patient. The deranged pathophysiology might enable individualized therapeutic regimens.

The invention provided herein allows personalized assessment of inflammatory diseases, and enables medical assessment of the distribution and severity of the disease or inflammation, the currently available treatments for the specific patient (this includes the underlying disease as well as the response to prior therapy or the concerns about potential contraindications due to other comorbidities), efficacy of the treatment (e.g., repeated assessments of the accompanying pathophysiology to see if a treatment responses is or is not as expected), and the current best estimate of outcome from the current treatment—a key prediction that can influence patient lifestyle choices.

One example of such medical assessment is illustrated by TNM staging for the common cancers, which is based upon the assessment of three components:
T—The extent of the primary tumor
N—The absence or presence and extent of regional lymph node metastasis
M—The absence or presence of distant metastasis

In TNM staging, numeric and alphanumeric subscripts are appended to each of these to provide a better description of the extent of the malignant tumor for each TNM category. The TNM is principally anatomic. Anatomic tumor indices form the basis for assessing cancer treatment response with methods such as RECIST. However, there is increasing recognition that tumor size change is slow and may not even reflect successful cancer cytostasis.

Yet, it is appreciated that pathophysiology is altered in all tumors and that changes in pathophysiology are likely to better reflect the progression of cancers and perhaps their response to treatment. Such pathophysiologic changes are often those due to the presence of Tumor Associated Macrophages (TAMs).

Surprisingly, nearly all forms of inflammation show the same neoplastic pathophysiology that is induced by TAMs except that, for inflammation, the inciting events can be much more diverse and the effector macrophages should be called Inflammation-associated Macrophages or IAMs. Inflammation that follows the accumulation of activated macrophages in the immediate region where the disease is active is imageable with the subject USPIOs nanoparticles using the methods and systems of the invention.

This dual assessment of local pathophysiology—vascularity and associated phagocytic activity of IAMs—are realizable with a single administration of the USPIO formulation and repeated imaging with MRI as appropriate. Magnetic resonance imaging is an imaging technology that is unusually sensitive to multiple disease processes and the information can be obtained without the use of ionizing radiation. There are a many useful MRI methods that can be used before or after USPIO administration and these additional utilities can provide added value. As described herein, in some circumstances ultrasound or optical imaging may replace or supplement MRI assessment of macrophage-dependent diseases.

The safety of non-invasive imaging is useful for the numerous times that a patient with inflammation could benefit from serial measurement of the extent and activity of his/her disease. Macrophage-dependent diseases, whether inflammation or neoplasia, can require repeated imaging and repeated exposure to ionizing radiation for diagnostic or evaluative purposes should be minimized.

Following intravenous administration of USIOs, there is a biodistribution and pharmacokinetic response that is similar for many USPIOs. The blood half-life and macrophage accumulation/retention times can differ. Since USPIOs are MR-imageable, they provide the ability to assess locally increased vascularity accompanying the macrophage-initiated angiogenesis and the locally increased phagocytosis of USPIO nanoparticles in IAMs can be used to address the questions posed above for all forms of chronic inflammation that are macrophage-dependent. These properties—MR visibility and the ability to assess both vascularity and macrophage activity—provide the surprising ability to characterize a diversity of inflammatory diseases following a single administration of the USPIO.

The methods and systems of the invention further provide direct therapeutic benefits. For example, the aggregated nanoparticles in tissue macrophages can facilitate biopsy or surgical removal of the disease tissues, or ablation of disease tissues via nanoparticle heating and/or targeted drug delivery with optional controlled drug release.

### EXAMPLES

### Example 1

### Experiments Assessing Macrophage Loading

Extensive preclinical research on a representative USPIO, ferumoxtran-10, shows the broad range of macrophage loading feasible in mammals. Intravenous doses ranging from 2-400 mg Fe/kg in rats and dogs show no significant adverse effects. Bourrinet (supra). These doses of ferumoxtran-10 are cleared from the blood by body macrophages within 24 hours. Any of the tissues with fixed macrophages could be used to determine the detection characteristics of USPIOs when they are aggregated in macrophages, using ultrasound or optical coherence imaging devices.

One particularly useful method uses percutaneous administration into the subcutaneous tissue on the dorsum of the foot in rabbits or rats see Wolf (Acad. Radiol., 6(1):55-60, 1999, incorporated herein by reference). Percutaneous lymphography relies upon the access of nanoparticles through the clefts in the terminal lymphatics. These single celled vessels are tethered to the surrounding stroma so that tissue movement (such as with exercise or massage) opens the gaps between the cells and facilitates nanoparticle entry. Once in the lymph, the nanoparticles are carried to the first draining lymph node where they are captured by macrophages lining the medullary sinuses.

Thus, percutaneous lymphography can be used to determine the detection sensitivity for imaging loaded macrophages. The important variables are the administered dose, the role of repeated doses, and the temporal removal of the delivered USPIO nanoparticles from the enhanced node. Multiple methods can be used to determine the amount of USPIO in the nodal macrophages. The change in T2* or susceptibility using MR is related to the USPIO concentration. Tissue analysis with Prussian Blue staining, staining the coating, and TEM can also be used as a comparison standard.

### Example 2

### Detection Sensitivity with Ultrasound (Macrophage-Enhanced Lymph Nodes: Method for Comparing the Imaging Detection and Characterization)

The rat or rabbit is injected with a challenge subcutaneous dose of USPIO, the injection site is gently massaged for 15 minutes, and then the animal is anesthetized for evaluation at an appropriate experimental time post injection. It is possible to use comparison substances injected in the same manner in the opposite hind paw.

The dose of UPSIO can be an experimental variable as well as the time after injection. These experiments allow the creation of loaded nodal macrophages where accumulation, retention, and metabolism can be evaluated. The experimental animals can be studied with any ultrasound device wherein ultrasound frequency, detection of backscatter, probe design, or other variable can be studied to determine detection parameters. Qualitative, semi-quantitative, or quantitative response can be obtained in reference to the in vivo MR or histologic data.

These experimental animals with variable loading of the nodal macrophages are then used in comparison imaging experiments.

Macrophage-enhanced nodes are imaged in vivo with MR, ultrasound, or optical devices. The sensitivity for detection, spatial resolution, and quantitation are determined versus the amount of USPIO present in the experimental node. Some animals can be serially studied. Finally, the animals can be euthanized and the nodes removed for in vitro studies or histopathology (see below).

### Example 3

### Detection Sensitivity with Optical Coherence Tomography

Using the methods above, OCT device variables and their detection sensitivity can be determined. The draining node in these animals is close to the surface, but surgical exposure can be used when experimentally appropriate.

### Example 4

### In Vitro Studies of Loaded or Control Lymph Nodes

Following the administration of the USPIO nanoparticles (by percutaneous lymphography, for example) and in vivo studies according to dose and time variables, as desired, the test subject can be euthanized, and the USPIO-enhanced and control nodes removed for in vitro studies. These nodes may be subject to histologic analysis, such as Prussian blue staining or TEM at the desired time. It may also be convenient to embed the experimental nodes in agar to create certain imaging variables such as distance from the ultrasound or OCT probe. Furthermore, unenhanced nodes can be dispersed with enhanced nodes in other agar volumes to determine imaging sensitivity and specificity.

Finally, there may be methods to fix the nodes so that they can be preserved for longer periods, such as suspending the nodes in tissue equivalent agar, such that the nodes can be preserved for repeated in vitro imaging comparisons. This may alter the MR dose-response curves.

Other tissues with fixed macrophages can be used to image detection sensitivity versus time, dose, and anatomic location. In this regard, both liver and spleen provide useful models for the response to intravenous administrations.

### Example 5

### Thermal Sensitivity

In vivo nodes or in vitro nodes are exposed to heating with an appropriate thermotherapy device that is effective for the depth of the enhanced node. The temperatures achieved versus USPIO concentration within the node or in surrounding unenhanced tissues or the nearby agar are determined. The relations between USPIO concentration, tissue location, and heating versus the delivered energy are constructed. These data are in turn used in heating models or with biothermal simulations to determine important responses and/or treatment parameters.

### Example 6

### Chemotherapy

The same animal models can be used to determine macrophage targeting with any USPIO+Drug formulation. It may be convenient to use nuclear medicine technologies where the iron, the coating, or the bound drug is tagged with appropriate isotopes.

### Example 7

### Chemothermotherapy

In like manner, the formulations that use pH or thermally sensitive release mechanisms can be used in similar experiments as described above.

### PUBLICATIONS

Ahsan F, Rivas I P, Khan M A, Tones Suarez A I, “Targeting to macrophages: role of physicochemical properties of particulate carriers—liposomes and microspheres—on the phagocytosis by macrophages,” J. Control Release, 79:29-40 (2002).
Bae Y H, Park K, “Targeted drug delivery to tumors: myths, reality and possibility,” J. Control Release, 153; 198-205 (2011).
Dabquah M K, Zhang X A, Mahato R I, “Extravasation of polymeric nanomedicines across tumor vasculature,” Adv. Drug Deliv. Rev., 63:628-639 (2011).
Porta C, Riboldi E, Totaro M G, Strauss L, Sica A, Mantofani A, “Macrophages in cancer and infectious diseases: the “good” and the “bad,” Immunotherapy, 10:1185-1202 (2011).
Maeda H, “Vascular permeability in cancer and infection as related to macromolecular drug delivery, with emphasis on the EPR effect for tumor-selective drug targeting,” Pro. Jpn. Acad. Ser. B. Phys. Biol. Sci., 88:53-71 (2012).
Waite C L, Roth C M, “Nanoscale drug delivery systems for enhanced drug penetration into solid tumors: current progress and opportunities,” Crit. Rev. Biomed. Eng., 40:21-41 (2012).
Levy M, “Degradability of superparamagnetic nanoparticles in a model of intracellular environment; follow-up of magnetic, structural and chemical properties,” Nanotechnology, 21:395103 (2010).
Matzinger P, “The Danger Model: A renewed sense of self,” Science, 296:302-305 (2002).
Matsushita T, et al., “Inflammatory imaging with ultrasmall superparamagnetic iron oxide,” Mag. Reson. Imaging, 29:171-178 (2011).
Corot C et al., “Recent advances in iron oxide nanocrystal technology for medical imaging,” Adv. Drug Del. Rev., 58: 1471-1504 (2006).
Levy M et al., “Long term in vivo biotransformation of iron oxide nanoparticle,” Biomaterials, 32:3988-3999 (2011).
Bohren C F, Huffman, D, Absorption and Scattering of Light by Small Particles. John Wiley, New York 1983.
Nolte I et al., “Iron particles enhanced visualization of experimental gliomas with high-resolution sonogaphy,” Am. J. Neuroradiol., 26:1469-1474 (2005).
Pober J S, Cotran R, “The role of endothelial cells in inflammation,” Transplantation, 50:537-544 (1990).
Huesinkveld M, van der Burg, S H, “Identification and manipulation of tumor-associated macrophages in human cancers,” J. Translat. Med., 9:216-240 (2011).
US 2011/0286938 Joshua; “Non-invasive detection of complement-mediated inflammation using CR2-targeted nanoparticles.”
US 2010/0166669 Diane; “Methods of imaging inflammation in pancreatic islets.”
US 2012/0003160 Wolf; “Macrophage-enhanced MRI (MEMRI) at single imaging session.”
US 2010/0305149 Yurkovetskly, “Polyal drug conjugates comprising variable rate-releasing linkers.”
US 2008/00318883 A1 Torchilin, “Condition-dependent multiple target delivery system.”
US 2010/0183504 Chen, “Multimodal imaging probes for in vivo targeted and non-targeted imaging and therapeutics.”
US 2011/0286938 Thurman, “Non-invasive detection of complement-mediated inflammation using CR2-targeted nanoparticles.”

All publications recited herein, including patents and patent application publications, are incorporated herein by reference.

1. A method for detecting activated macrophages in a subject, wherein the subject has a disease or condition associated with said activated macrophages, the method comprising:
(1) administering to the subject a formulation of ultrasmall superparamagnetic iron oxide (USPIO) nanoparticles having an average size of about 10-50 nm;
(2) waiting for a pre-determined time to allow the USPIO nanoparticles to accumulate as aggregates inside said activated macrophages, wherein the aggregates have an average size of greater than 100 nm; and,
(3) imaging the activated macrophages with a medical imaging device that operates at a wavelength between 0.1 μm and 1 m, and produces an image based at least in part on Rayleigh and/or Mie scattering by the aggregates;

2. The method of claim 1, further comprising assessing the location, density, and/or degree of locally enhanced activated macrophages, thereby staging the macrophage associated disease or condition.

3. The method of claim 1, further comprising evaluating the image to determine (1) the need for treatment, (2) treatment options using the USPIO-enhanced macrophages for treating the disease existing at one or more sites comprising said activated macrophages, at the time of evaluation, and/or (3) determining the need for further USPIO dosing regimens to make such treatments feasible.

4. A method for treating a disease or condition associated with activated macrophages, in a subject in need thereof, the method comprising:
(1) administering to the subject a formulation of an ultrasmall superparamagnetic iron oxide (USPIO) nanoparticles, wherein the USPIO nanoparticles have an average size of about 10-50 nm;
(2) waiting for a pre-determined time to allow the USPIO nanoparticles to accumulate as aggregates in said activated macrophages, wherein the aggregates have an average size of greater than 100 nm;
(3) locating the activated macrophages with a medical imaging device to identify the size, shape, and/or location of tissues affected by the disease or condition;
(4) raising temperature of the aggregates by directing an energy that is absorbed by the aggregates, and sustaining the elevated temperature for a time sufficient to kill the cells in tissues affected by the disease or condition and surrounding said activated macrophage.

5. The method of claim 4, wherein the USPIO nanoparticles are coated with a therapeutic agent effective for treating said disease or condition.

6. The method of claim 5, wherein the therapeutic agent is released upon raising the temperature of the aggregates.

7. The method of claim 4, wherein the energy is high intensity ultrasound energy.

8. The method of claim 4, further comprising assessing macrophage density during therapeutic intervention in order to determine the desirable continuation, change, or cessation of a particular therapy.

9. The method of claim 4, wherein the USPIO nanoparticles have an average hydrodynamic particle size of about 15-50 nm as determined by dynamic light scattering.

10. The method of claim 4, wherein the formulation of USPIO nanoparticles is administered to the subject percutaneously, intravenously, by inhalation or ingestion, or otherwise into a body cavity connected to the outside.

11. The method of claim 4, wherein the formulation of USPIO nanoparticles is administered to the subject:
(1) intravenously at a dose of about 0.5 to 20 mg Fe/kg, optionally repeated (as necessary) with a waiting time between 12 and 144 hours or longer;
(2) interstitially (for nodal enhancement) at a dose of about 0.01-2 mg Fe/kg, with stimulation of lymphatic uptake as feasible, and, optionally repeated (as necessary) with a waiting time of between 30 min-14 days; or,
(3) intracavitarily at a dose of about 0.05-2 mg Fe/kg (in an appropriate suspension), and waiting 30 min-14 days before imaging.

12. The method of claim 4, wherein the pre-determined time is sufficient to allow the USPIO nanoparticles to accumulate as aggregates of at least about 100 nm in size, or 1 μm in size, or 1.5 μm in size, or 2 μm in size, preferably occupying 5-80% of the macrophage cell volume.

13. The method of claim 4, further comprising controlling the size of the aggregates by dose, administration route, waiting time, and frequency of the USPIO formulation administered to said subject.

14. The method of claim 4, wherein the iron core of the USPIO nanoparticles are coated by a biocompatible polymer.

15. The method of claim 4, wherein said medical imaging device is ultrasound or optical imaging.
